(19) United States
(12) Patent Application Publication
(10) Pub. No.: US 2024/0287427 A1
(43) Pub. Date: Aug. 29, 2024

(54) DEVICE AND METHOD FOR PREPARING COMPARTMENTALIZED IN VITRO MODELS WITH AN ELONGATED COMPONENT OF A BIOLOGICAL MATERIAL

(71) Applicant: 9493662 CANADA INC., Montreal (CA)

(72) Inventors: Subin Mac George, Orleans (CA); Margaret Haiganouch Magdesian, Saint-Laurent (CA)

(21) Appl. No.: 18/571,065

(22) PCT Filed: Jun. 16, 2022
(86) PCT No.: PCT/CA2022/050967

§ 371 Date: Dec. 15, 2023

Related U.S. Application Data
(60) Provisional application No. 63/212,308, filed on Jun. 18, 2021.

(51) Int. Cl. C12M 3/00; C12M 1/00; C12M 1/42

(52) U.S. Cl. CPC C12M 21/08; C12M 23/34; C12M 23/38; C12M 23/40; C12M 29/04; C12M 35/02

(57) ABSTRACT

There are provided cell culture devices and associated methods for preparing a compartmentalized in vitro model. The cell culture device can include a cell culture layer that includes an inlet reservoir configured for receiving an inlet fluid medium therein, an outlet reservoir configured for receiving an outlet fluid medium therein, and a channel extending between the inlet reservoir and the outlet reservoir to establish fluid communication therebetween. The channel can include a multicellular component receiving portion and an elongated component receiving portion provided downstream of the multicellular component receiving portion, the elongated component receiving portion being sized and configured for orienting an elongated component of a biological material away from the inlet reservoir. The cell culture device can further include an electrode layer provided in proximity of the cell culture layer.

### TECHNICAL FIELD

The technical field generally relates to cell culture techniques. More particularly, the technical field relates to cell culture techniques for preparing compartmentalized in vitro models with a biological material that includes an elongated component.

### BACKGROUND

Models that can predict human or animal responses to various chemical and biological compounds, such as medications, pesticides, and cosmetics, and that are easily scalable to test multiple compounds already on the market and new products launched each year are desirable.

In vitro models can be used to refine, reduce, and replace animal testing, for instance in the pharmaceutical, chemical and cosmetic industries. In vitro models can offer opportunities for performing various experiments on biological materials. These experiments can be performed for instance in relation to toxicity testing, efficacy testing, drug screening, drug repurposing, disease modelling, etc.

However, conventional techniques for modeling biological tissues in vitro do not enable compartmentalization of components of a biological tissue or do not enable obtaining an organized architecture of the components of the biological tissue. For instance, hair or neurons are typically grown according to an unorganized network, which can inaccurately model such biological materials and render the model unsuitable for performing certain tests on specific components of the biological material.

Accordingly, there remain a number of challenges with respect to the production of in vitro models of biological materials.

### SUMMARY

In accordance with an aspect, there is provided a cell culture device for preparing a compartmentalized in vitro model using a biological material having a multicellular component and an elongated component, the cell culture device comprising:
a cell culture layer couplable to a reservoir of a cell culture plate, the cell culture layer comprising:
   an inlet reservoir configured for receiving an inlet fluid medium therein;
   an outlet reservoir configured for receiving an outlet fluid medium therein; and
   a channel extending between the inlet reservoir and the outlet reservoir to establish fluid communication therebetween, the channel comprising:
      a multicellular component receiving portion sized and configured for receiving at least a portion of the multicellular component of the biological material; and
      an elongated component receiving portion provided downstream of the multicellular component receiving portion, the elongated component receiving portion being sized and configured for orienting the elongated component of the biological material away from the inlet reservoir and for preventing the multicellular component from travelling past the multicellular component receiving portion.

In some implementations, the channel comprises a converging portion that converges inwardly toward the outlet reservoir.

In some implementations, the converging portion defines at least a portion of the multicellular component receiving portion of the channel.

In some implementations, the converging portion defines at least a portion of the elongated component receiving portion of the channel.

In some implementations, the converging portion includes an inwardly converging top wall that converges inwardly toward a center of the channel.

In some implementations, the inwardly converging top wall defines a step change.

In some implementations, the inwardly converging top wall includes a curvature.

In some implementations, the channel has a width that is substantially constant.

In some implementations, the converging portion includes an inwardly converging sidewall that converges inwardly toward a center of the channel.

In some implementations, the inwardly converging sidewall defines a step change.

In some implementations, the inwardly converging sidewall includes a curvature.

In some implementations, the converging portion comprises a frustoconical converging portion.

In some implementations, the converging portion comprises a frustopyramidal converging portion.

In some implementations, the converging portion comprises a neck portion to contribute to stabilizing the elongated component of the biological material.

In some implementations, the converging portion comprises an inwardly protruding member to contribute to stabilizing the elongated component of the biological material.

In some implementations, the channel further comprises a tubular portion provided downstream of the converging portion, the tubular portion having a substantially constant diameter throughout its length.

In some implementations, the converging portion of the channel comprises converging sections each converging at a corresponding angle toward the outlet reservoir.

In some implementations, the converging sections successively comprises a first converging section and a second converging section, the corresponding angle of the first converging section being larger than the corresponding angle of the second converging section.

In some implementations, a transition between successive ones of the converging sections comprises a curved transition.

In some implementations, a transition between successive ones of the converging sections comprises a sharp transition.

In some implementations, the channel is sized and configured to maintain the elongated component of the biological material in a substantially elongated configuration.

In some implementations, the channel comprises a plurality of channels.

In some implementations, adjacent channels of the plurality of channels are similar to each other.

In some implementations, at least one channel of the plurality of channels is configured differently than at least one other channel of the plurality of channels.

In some implementations, the inlet reservoir comprises a plurality of inlet reservoirs.

In some implementations, at least one channel of the plurality of channels is in fluid communication with a corresponding inlet reservoir of the plurality of inlet reservoirs.

In some implementations, the outlet reservoir comprises a plurality of outlet reservoirs.

In some implementations, at least one channel of the plurality of channels is in fluid communication with a corresponding outlet reservoir of the plurality of outlet reservoirs.

In some implementations, the cell culture layer is configured to extend substantially horizontally, the inlet reservoir and the outlet reservoir being provided in a longitudinally spaced-apart relationship.

In some implementations, the cell culture layer further comprises an inlet well in fluid communication with the inlet reservoir, the inlet well being configured to receive the inlet fluid medium therein.

In some implementations, the cell culture layer comprises an inlet manifold extending between the inlet well and the inlet reservoir, the inlet manifold comprising a plurality of inlet reservoir channels for directing flow of the inlet fluid medium from the inlet well to the inlet reservoir with reduced turbulence.

In some implementations, the cell culture layer comprises an inlet reservoir channel extending between the inlet well and the inlet reservoir for directing flow of the inlet fluid medium into the inlet reservoir with reduced turbulence.

In some implementations, the inlet reservoir channel is a converging inlet reservoir channel converging toward the inlet reservoir.

In some implementations, the inlet reservoir channel comprises at least one turbulence reducing feature.

In some implementations, the cell culture layer further comprises an outlet well in fluid communication with the outlet reservoir, the outlet well being configured to receive the outlet fluid medium therein.

In some implementations, the cell culture layer comprises an outlet manifold extending between the outlet reservoir and the outlet well, the outlet manifold comprising a plurality of outlet reservoir channels for directing flow of the outlet fluid medium from the outlet reservoir to the outlet well with reduced turbulence.

In some implementations, the cell culture layer comprises an outlet reservoir channel extending between the outlet reservoir and the outlet well for directing flow of the outlet fluid medium from the outlet reservoir to the outlet well with reduced turbulence.

In some implementations, the outlet reservoir channel is a converging outlet reservoir channel converging toward the outlet well.

In some implementations, the outlet reservoir channel comprises at least one turbulence reducing feature.

In some implementations, the cell culture layer is configured to extend substantially horizontally, the inlet reservoir and the outlet reservoir being provided in a superposed relationship, and the converging portion being a downwardly converging portion.

In some implementations, the outlet reservoir is U-shaped.

In some implementations, the inlet reservoir comprises a plurality of inlet reservoirs, and adjacent ones of the plurality of inlet reservoirs are in fluid communication via an inlet reservoir bridge extending therebetween.

In some implementations, the cell culture device further comprises a cover configured to be positionable on an upper surface of the cell culture layer.

In some implementations, the cover is configured to provide a fluid tight closure for the inlet reservoir once positioned on the upper surface of the cell culture layer.

In some implementations, the cover comprises a microporous membrane.

In some implementations, the cover comprises a collagen membrane.

In some implementations, the cell culture device further comprises a biological model positionable on an upper surface of the cell culture layer.

In some implementations, the biological model is positionable on the cell culture layer to enable interaction with the biological material received in the channel.

In some implementations, the biological model comprises cultured cells.

In some implementations, the biological model comprises a biological tissue.

In some implementations, the biological model comprises a biological tissue model.

In some implementations, the biological tissue model comprises a three-dimensional skin model.

In some implementations, the cell culture device further comprises an inlet well feeding system in fluid communication with the inlet well to supply additional fluid culture medium to the inlet well.

In some implementations, the cell culture plate is a petri dish.

In some implementations, the cell culture device complies with American National Standards Institute of the Society for Laboratory Automation and Screening (ANSI/SLAS) microplate standards.

In some implementations, the multicellular component of the biological material comprises a cluster of neuronal cell bodies and the elongated component comprising axons.

In some implementations, the multicellular component of the biological material comprises a follicle and the elongated component comprising a hair.

In accordance with another aspect, there is provided a cell culture device for preparing a compartmentalized in vitro model using a biological material having a multicellular component and an elongated component, the cell culture device comprising:
a cell culture layer insertable in a reservoir of a cell culture plate, the cell culture layer comprising:
   an inlet reservoir configured for receiving an inlet fluid medium therein;
   an outlet reservoir configured for receiving an outlet fluid medium therein; and
   a channel extending between the inlet reservoir and the outlet reservoir to establish fluid communication therebetween, the channel comprising:
      a multicellular component receiving portion sized and configured for receiving at least a portion of the multicellular component of the biological material; and
      an elongated component receiving portion provided downstream of the multicellular component receiving portion, the elongated component receiving portion being sized and configured for orienting the elongated component of the biological material away from the inlet reservoir and for preventing the multicellular component from travelling past the multicellular component receiving portion; and an electrode provided in proximity of the cell culture layer.

In some implementations, the electrode forms part of an electrode layer.

In some implementations, the electrode layer is positionable underneath the cell culture layer.

In some implementations, the electrode layer is receivable onto an upper surface of the cell culture layer.

In some implementations, the cell culture device further comprises a biological model receivable on an upper surface of the cell culture layer.

In some implementations, the electrode layer is provided onto the biological model.

In some implementations, the electrode layer is provided as part of the biological model.

In some implementations, the electrode comprises a plurality of electrodes.

In some implementations, the channel comprises a plurality of channels.

In some implementations, the plurality of electrodes are distributed over the electrode layer in accordance with a configuration of the plurality of channels.

In some implementations, the electrode is located in an adjacent reservoir.

In some implementations, the electrode comprises at least one of a metallic electrode, a metal oxide electrode, a carbon electrode, a multi-electrode array, and a field effect transistor detector.

In some implementations, the electrode is configured for stimulating the biological material.

In some implementations, the electrode is configured for at least one of collecting, recording, measuring, or detecting a response of the biological material to stimulation.

In some implementations, the cell culture device further comprises an electronic device in ohmic connection with the electrode.

In some implementations, the electronic device comprises a sensing device.

In some implementations, the electronic device comprises a stimulating device.

In some implementations, the electronic device is configured for providing an electrical read-out comprising at least one of a potential recording, an impedance spectroscopy recording, a voltammetry recording and an amperometry recording.

In some implementations, the cell culture device further comprises a sensor configured for stimulating the biological material, measuring a response from the biological material to stimulation, providing an outlet or receiving an inlet.

In some implementations, the sensor comprises an optical or an electrical transducer.

In accordance with another aspect, there is provided a method for preparing a compartmentalized in vitro model of a biological material that includes a multicellular component and an elongated component using a cell culture layer receivable in a cell culture plate, the method comprising:
supplying a fluid culture medium to an inlet reservoir of the cell culture layer;
positioning the multicellular component of the biological material within the inlet reservoir of the cell culture plate; and
inserting and/or growing at least a portion of the elongated component of the biological material into a channel of the cell culture layer that is in fluid communication with the inlet reservoir, the channel comprising a multicellular component receiving portion and an elongated component receiving portion provided downstream of the multicellular component receiving portion, the elongated component receiving portion being sized and configured for orienting the elongated component of the biological material away from the inlet reservoir and for preventing the multicellular component from travelling past the multicellular component receiving portion.

In some implementations, the channel comprises a converging portion that converges inwardly.

In some implementations, the cell culture layer further comprises an outlet reservoir in fluid communication with the channel, with the channel extending between the inlet reservoir and the outlet reservoir.

In some implementations, at least a portion of the elongated component of the biological material extend within the outlet reservoir.

In some implementations, the method further comprises adding an outlet test substance to the outlet reservoir to perform testing on the at least a portion of the elongated component of the biological material.

In some implementations, the method further comprises positioning an additional biological material within the outlet reservoir.

In some implementations, the method further comprises adding an outlet test substance to the outlet reservoir to perform testing on additional biological material.

In some implementations, the method further comprises adding an inlet test substance to the inlet reservoir to perform testing on the non-elongated component of the biological material.

In some implementations, the method further comprises placing a cover on an upper surface of the cell culture layer.

In some implementations, the cover comprises a microporous membrane.

In some implementations, the cover comprises a collagen membrane.

In some implementations, the method further comprises placing a biological model on an upper surface of the cell culture layer.

In some implementations, the biological model is positionable on the cell culture layer to enable interaction with the biological material received in the cell culture layer.

In some implementations, the multicellular component of the biological material comprises follicles.

In some implementations, the elongated component of the biological material comprises hair.

In some implementations, the multicellular component of the biological material comprises cell bodies of neurons.

In some implementations, the biological material is provided as a neurosphere.

In some implementations, the elongated component of the biological material comprises axons.

In accordance with another aspect, there is provided a method for preparing a compartmentalized in vitro model of a biological material using a cell culture layer receivable in a cell culture plate, the method comprising:
supplying a fluid culture medium to an inlet reservoir of the cell culture layer;
positioning a first biological material that includes a multicellular component within the inlet reservoir of the cell culture plate; and
inserting and/or growing a second biological material into a channel of the cell culture layer that is in fluid communication with the inlet reservoir, the second biological material comprising an elongated component biologically interacting with the first biological material, and the channel being configured to maintain the elongated component in a substantially elongated configuration.

In some implementations, the channel comprises a converging portion that converges inwardly toward the outlet reservoir.

In some implementations, the cell culture layer further comprises an outlet reservoir in fluid communication with the channel, with the channel extending between the inlet reservoir and the outlet reservoir.

In some implementations, at least a portion of the second biological material extends within the outlet reservoir.

In some implementations, the method further comprises adding an outlet test substance to the outlet reservoir to perform testing on the second biological material.

In some implementations, the method further comprises positioning an additional biological material within the outlet reservoir.

In some implementations, the method further comprises adding an outlet test substance to the outlet reservoir to perform testing on the additional biological material.

In some implementations, the method further comprises adding an inlet test substance to the inlet reservoir to perform testing on the first biological material.

In some implementations, the method further comprises placing a cover on an upper surface of the cell culture layer.

In some implementations, the cover comprises a microporous membrane.

In some implementations, the cover comprises a collagen membrane.

In some implementations, the method further comprises placing a biological model on an upper surface of the cell culture layer.

In some implementations, the biological model is positionable on the cell culture layer to enable interaction with the biological material received in the cell culture layer.

In some implementations, the first biological material comprises neurons.

In some implementations, the neurons are provided as neurospheres.

In some implementations, the elongated component of the second biological material comprises axons.

In some implementations, the first biological material comprises follicles.

In some implementations, the second biological material comprises hairs.

In some implementations, the additional biological material comprises at least one of heart tissue, intestinal tissue, muscle tissue, and corneal tissue.

In accordance with another aspect, there is provided a cell culture device for preparing a compartmentalized in vitro model using a biological material having a multicellular component and an elongated component, the cell culture device comprising:
a cell culture layer couplable to a reservoir of a cell culture plate, the cell culture layer comprising:
   an inlet reservoir configured for receiving an inlet fluid medium and the multicellular component of the biological material therein;
   an outlet reservoir configured for receiving an outlet fluid medium therein; and
   a channel extending between the inlet reservoir and the outlet reservoir to establish fluid communication therebetween, the channel being sized and configured for selectively retaining the multicellular component of the biological material upstream of the channel while the elongated component extends within the channel.

In some implementations, the channel comprises a converging portion that converges inwardly toward the outlet reservoir.

In some implementations, the converging portion includes an inwardly converging top wall that converges inwardly toward a center of the channel.

In some implementations, the inwardly converging top wall defines a step change.

In some implementations, the inwardly converging top wall includes a curvature.

In some implementations, the channel has a width that is substantially constant.

In some implementations, the converging portion includes an inwardly converging sidewall that converges inwardly toward a center of the channel.

In some implementations, the inwardly converging sidewall defines a step change.

In some implementations, the inwardly converging sidewall includes a curvature.

In some implementations, the converging portion comprises a frustoconical converging portion.

In some implementations, the converging portion comprises a frustopyramidal converging portion.

In some implementations, the converging portion comprises a neck portion to contribute to stabilizing the elongated component of the biological material.

In some implementations, the converging portion comprises an inwardly protruding member to contribute to stabilizing the elongated component of the biological material.

In some implementations, the channel further comprises a tubular portion provided downstream of the converging portion, the tubular portion having a substantially constant diameter throughout its length.

In some implementations, the converging portion of the channel comprises converging sections each converging at a corresponding angle toward the outlet reservoir.

In some implementations, the converging sections successively comprises a first converging section and a second converging section, the corresponding angle of the first converging section being larger than the corresponding angle of the second converging section.

In some implementations, a transition between successive ones of the converging sections comprises a curved transition.

In some implementations, a transition between successive ones of the converging sections comprises a sharp transition.

In some implementations, the channel is sized and configured to maintain the elongated component of the biological material in a substantially elongated configuration.

In some implementations, the channel comprises a plurality of channels.

In some implementations, adjacent channels of the plurality of channels are similar to each other.

In some implementations, at least one channel of the plurality of channels is configured differently than at least one other channel of the plurality of channels.

In some implementations, the inlet reservoir comprises a plurality of inlet reservoirs.

In some implementations, at least one channel of the plurality of channels is in fluid communication with a corresponding inlet reservoir of the plurality of inlet reservoirs.

In some implementations, the outlet reservoir comprises a plurality of outlet reservoirs.

In some implementations, at least one channel of the plurality of channels is in fluid communication with a corresponding outlet reservoir of the plurality of outlet reservoirs.

In some implementations, the cell culture layer is configured to extend substantially horizontally, the inlet reservoir and the outlet reservoir being provided in a longitudinally spaced-apart relationship.

In some implementations, the cell culture layer further comprises an inlet well in fluid communication with the inlet reservoir, the inlet well being configured to receive the inlet fluid medium therein.

In some implementations, the cell culture layer comprises an inlet manifold extending between the inlet well and the inlet reservoir, the inlet manifold comprising a plurality of inlet reservoir channels for directing flow of the inlet fluid medium from the inlet well to the inlet reservoir with reduced turbulence.

In some implementations, the cell culture layer comprises an inlet reservoir channel extending between the inlet well and the inlet reservoir for directing flow of the inlet fluid medium into the inlet reservoir with reduced turbulence.

In some implementations, the inlet reservoir channel is a converging inlet reservoir channel converging toward the inlet reservoir.

In some implementations, the inlet reservoir channel comprises at least one turbulence reducing feature.

In some implementations, the cell culture layer further comprises an outlet well in fluid communication with the outlet reservoir, the outlet well being configured to receive the outlet fluid medium therein.

In some implementations, the cell culture layer comprises an outlet manifold extending between the outlet reservoir and the outlet well, the outlet manifold comprising a plurality of outlet reservoir channels for directing flow of the outlet fluid medium from the outlet reservoir to the outlet well with reduced turbulence.

In some implementations, the cell culture layer comprises an outlet reservoir channel extending between the outlet reservoir and the outlet well for directing flow of the outlet fluid medium from the outlet reservoir to the outlet well with reduced turbulence.

In some implementations, the outlet reservoir channel is a converging outlet reservoir channel converging toward the outlet well.

In some implementations, the outlet reservoir channel comprises at least one turbulence reducing feature.

In some implementations, the cell culture layer is configured to extend substantially horizontally, the inlet reservoir and the outlet reservoir being provided in a superposed relationship, and the converging portion being a downwardly converging portion.

In some implementations, the outlet reservoir is U-shaped.

In some implementations, the inlet reservoir comprises a plurality of inlet reservoirs, and adjacent ones of the plurality of inlet reservoirs are in fluid communication via an inlet reservoir bridge extending therebetween.

In some implementations, the cell culture device further comprises a cover configured to be positionable on an upper surface of the cell culture layer.

In some implementations, the cover is configured to provide a fluid tight closure for the inlet reservoir once positioned on the upper surface of the cell culture layer.

In some implementations, the cover comprises a microporous membrane.

In some implementations, the cover comprises a collagen membrane.

In some implementations, the cell culture device further comprises a biological model positionable on an upper surface of the cell culture layer.

In some implementations, the biological model is positionable on the cell culture layer to enable interaction with the biological material received in the channel.

In some implementations, the biological model comprises cultured cells.

In some implementations, the biological model comprises a biological tissue.

In some implementations, the biological model comprises a biological tissue model.

In some implementations, the biological tissue model comprises a three-dimensional skin model.

In some implementations, the cell culture device further comprises an inlet well feeding system in fluid communication with the inlet well to supply additional fluid culture medium to the inlet well.

In some implementations, the cell culture plate is a petri dish.

In some implementations, the cell culture device complies with American National Standards Institute of the Society for Laboratory Automation and Screening (ANSI/SLAS) microplate standards.

In some implementations, the multicellular component of the biological material comprises a cluster of neuronal cell bodies and the elongated component comprising axons.

In some implementations, the multicellular component of the biological material comprises a follicle and the elongated component comprising a hair.

In accordance with another aspect, there is provided a method for preparing a compartmentalized in vitro model of a biological material that includes a multicellular component and an elongated component using a cell culture layer receivable in a cell culture plate, the method comprising:
supplying a fluid culture medium to an inlet reservoir of the cell culture layer;
positioning the multicellular component of the biological material within a multicellular component receiving portion of a channel of the cell culture layer, the channel being in fluid communication with the inlet reservoir; and
inserting and/or growing the elongated component of the biological material into an elongated component receiving portion of the channel located downstream of the multicellular component receiving portion, the elongated component receiving portion of the channel being sized and configured for preventing the multicellular component from travelling past the multicellular component receiving portion.

In some implementations, the channel comprises a converging portion that converges inwardly.

In some implementations, the cell culture layer further comprises an outlet reservoir in fluid communication with the channel, with the channel extending between the inlet reservoir and the outlet reservoir.

In some implementations, at least a portion of the elongated component of the biological material extend within the outlet reservoir.

In some implementations, the method further comprises adding an outlet test substance to the outlet reservoir to perform testing on the at least a portion of the elongated component of the biological material.

In some implementations, the method further comprises positioning an additional biological material within the outlet reservoir.

In some implementations, the method further comprises adding an outlet test substance to the outlet reservoir to perform testing on additional biological material.

In some implementations, the method further comprises adding an inlet test substance to the inlet reservoir to perform testing on the non-elongated component of the biological material.

In some implementations, the method further comprises placing a cover on an upper surface of the cell culture layer.

In some implementations, the cover comprises a microporous membrane.

In some implementations, the cover comprises a collagen membrane.

In some implementations, the method further comprises placing a biological model on an upper surface of the cell culture layer.

In some implementations, the biological model is positionable on the cell culture layer to enable interaction with the biological material received in the cell culture layer.

In some implementations, the multicellular component of the biological material comprises follicles.

In some implementations, the elongated component of the biological material comprises hair.

In some implementations, the multicellular component of the biological material comprises cell bodies of neurons.

In some implementations, the biological material is provided as a neurosphere.

In some implementations, the elongated component of the biological material comprises axons.

In some implementations, the method further comprises one or more features as defined herein and/or as described and/or illustrated herein.

### BRIEF DESCRIPTION OF THE DRAWINGS

The attached figures illustrate various features, aspects and implementations of the technology described herein.

FIG. 1 is a perspective view of a cell culture device in accordance with an implementation, the cell culture device including an inlet reservoir, an outlet reservoir, and a plurality of channels extending between the inlet reservoir and the outlet reservoir.

FIG. 2 is a top view of the cell culture device of FIG. 1.

FIG. 3 is a top view of a cell culture device in accordance with another implementation, the cell culture device including two units each having an inlet reservoir, an outlet reservoir, and a plurality of channels extending between the inlet reservoir and the outlet reservoir.

FIG. 4 is a perspective view of the cell culture device of FIG. 3, placed onto a bottom wall of a cell culture plate and shown with a cover.

FIG. 5 is a top view of the cell culture device and cell culture plate of FIG. 4.

FIGS. 6A and 6B are top views of a portion of the cell culture device of FIG. 3, showing the plurality of channels having a similar configuration.

FIGS. 7A and 6B are top views of a portion of a cell culture device in accordance with another implementation, the cell culture device including two units each having an inlet reservoir, an outlet reservoir, and a plurality of channels extending between the inlet reservoir and the outlet reservoir, and the plurality of channels having various configurations.

FIG. 8 is a cross-sectional perspective view of the cell culture device of FIG. 1.

FIG. 9 is a portion of the cross-sectional view of FIG. 8, showing a channel that includes an elongated component receiving portion that includes a converging portion having a first converging section and a second converging section.

FIG. 10 is a portion of the perspective view of the cell culture device of FIG. 1.

FIG. 11 is a schematic representation of a top view of a channel of the plurality of channel of the cell culture device of FIG. 1.

FIG. 12 is a schematic representation of a side view of the channel of FIG. 11.

FIG. 13 is a cross-sectional perspective view of a portion of the perspective view of the cell culture device of FIG. 3.

FIG. 14 is a schematic representation of a side view of a channel of the plurality of channels of the cell culture device of FIG. 3.

FIG. 15 is a top view of a cell culture device in accordance with another implementation, the cell culture device including two units each having an inlet reservoir, an outlet reservoir, and a plurality of channels extending between the inlet reservoir and the outlet reservoir.

FIG. 16 is a top view of a portion of the cell culture device of FIG. 15, showing the plurality of channels of one of the units having a similar configuration.

FIG. 17 is a top view of a portion of the cell culture device of FIG. 15, showing the plurality of channels of another one of the units having a similar configuration.

FIG. 18 is a cross-sectional perspective view of the cell culture device of FIG. 15 and top view of the top view of a portion of the cell culture device of FIG. 15, showing the plurality of channels of another one of the units having a similar configuration.

FIG. 18A is a top view of a portion of the cell culture device of FIG. 18.

FIG. 19 is a cross-sectional side view of a portion of the cell culture device of FIG. 18, showing a channel having a multicellular component receiving portion and an elongated component receiving portion.

FIG. 20 is a top view of a cell culture device in accordance with another implementation, the cell culture device including two units each having an inlet reservoir, an outlet reservoir, and a plurality of channels extending between the inlet reservoir and the outlet reservoir.

FIG. 21 is a cross-sectional view of a channel of the plurality of channels of the cell culture device of FIG. 20.

FIG. 22 is a perspective view of a cell culture device in accordance with another implementation, the cell culture device including three pairs of inlet reservoirs, with a corresponding outlet reservoir being provided underneath each pair of inlet reservoirs, the two inlet reservoirs of each pair of inlet reservoirs being placed in fluid communication via a corresponding inlet reservoir bridge that extends therebetween.

FIG. 23 is a top view of the cell culture device of FIG. 22.

FIG. 24 is a cross-sectional view of the cell culture device of FIG. 22.

FIG. 25 is another cross-section view of the cell culture device of FIG. 22.

FIG. 26 is a cross-sectional view of a portion of the cell culture device of FIG. 22, showing a channel that includes an elongated component receiving portion.

FIG. 27 is a perspective view of a cell culture device that includes a cell culture layer and an electrode layer.

FIG. 28 is a top view of the cell culture device of FIG. 27.

### DETAILED DESCRIPTION

Techniques described herein relate to the development of compartmentalized in vitro models, i.e., in vitro models of a biological material that includes a multicellular component and an elongated component, the elongated component being cultured according to a given architecture such that the elongated component can maintain its elongated configuration during growth and subsequent testing, as well as extend and grow extensions inside one or more environments. An example of such compartmentalized in vitro model is a hair that extends from a hair follicle grown in a liquid environment that is compatible with culture of cells present in the hair follicle, with the hair being exposed to an air environment. Maintaining an elongated configuration of the elongated component of the biological material can contribute to more accurately mimic in vivo behavior of the biological material, and can also enable performing distinct testing on a given component of the biological material while maintaining a biological interaction between the respective components of the biological material. In addition, compartmentalized in vitro models as described herein can enable two or more types of biological material to be placed in sufficiently close proximity to enable the biological interaction therebetween to be modelled.

As used herein, the expression “multicellular component” can refer to multicellular structures such as tissues, organs, organisms, organoids, spheroids, neurospheroids, and any other biological structure or clusters or cells where two or more cells, of same cell type or different cell types, are assembled.

A biological material that can be modeled using compartmentalized in vitro models can include a particular cell type, such as neurons, or can include multiple cell types, for examples neurons and glia. It can also include specific structures such as follicles and hairs. In these examples, the axons of the neurons and the hairs can each be considered as an elongated component of a biological material. Such biological material can also be combined with other biological materials to further mimic certain complex biological tissues and produce for instance innervated skin, skin with hairs, innervated intestines, innervated heart, innervated muscle, innervated cornea, etc. Thus, the compartmentalized in vitro models can be obtained for a biological material that includes an elongated material and at least another structure that are each being compartmentalized, such as neurons that include cell bodies, and axons as an elongated component, or a hair that includes a follicle and the hair itself as the elongated component, or between two biological materials with at least one of them including an elongated component, such as between heart tissue and neurons. Cells of various other organs can also be modelled.

The compartmentalized in vitro models as described herein can be used for a wide range of cellular assays including compound screening, compound discovery, safety, efficacy testing, etc. The organized architecture of a biological material or biological materials that can be obtained by implementing the compartmentalized in vitro model as described herein can offer multiple opportunities for performing various tests on a biological material cultured according to an in vitro model that more closely resemble the characteristics of a given animal or human biological tissue compared to conventional non-compartmentalized in vitro models.

The techniques described herein in relation to the preparation of compartmentalized in vitro models involve a cell culture device that includes a cell culture layer that can be inserted into a receptacle of a cell culture plate such as those that are widely available on the market. The cell culture layer can take various forms, and generally includes designated compartments each configured for receiving a given component of a biological material or a given biological material.

For instance, the compartmentalized in vitro model can include distinct reservoirs that are in fluid communication with each other via at least one channel, the channel being configured for orienting an elongated component of the biological material or of one of the biological materials. In some implementations, the channels can also be configured to contribute to maintaining the elongated component in an elongated configuration.

The reservoirs can enable contact of a given component of the biological material with a test substance, which can be the same or be different depending on the reservoir. A response of the given component of the biological material to the test substance can then be analyzed. The presence of distinct reservoirs can facilitate the analysis of a response from each component of the biological material to a test substance, and the analysis of the interaction of the components of the biological material to one or more test substances.

Furthermore, when the compartmentalized in vitro model includes two types of biological materials, such as neurons and skin, or neurons and muscles, for instance, each of the biological materials can be tested with a test substance, which can be the same or be different depending on the reservoir, and a response of each of the biological materials to the one or more test substances can be analyzed. The interaction between the biological materials in response to the one or more test substances can also be analyzed.

The use of such compartmentalized in vitro models can enable the generation of predictive data of compounds' safety and efficacy prior to exposure to humans, and can enable the pharmaceutical, chemical, and cosmetic industries to perform reproducible and faster drug screening, toxicity, and efficacy testing and in a more cost-effectively approach compared to conventional technologies. The miniaturization of tests performed using a compartmentalized in vitro model as described herein can also enable reducing the use of reagents per experiment. Furthermore, high throughput (HT) testing of multiple compounds using a compartmentalized in vitro model as described herein can facilitate clinical translatability, thereby predicting the efficacy and toxicity of compounds faster and more efficiently.

It will be appreciated that positional descriptions such as “above”, “below”, “left”, “right”, “inwardly”, “outwardly”, “vertical” and the like should, unless otherwise indicated, be taken in the context of the figures, and should not be considered limiting. When referring to a length, for instance in the context of a length of an axon, it is to be understood that it refers to a measure along a horizontal axis. When referring to a height, for instance in the context of a height of a channel of a microfluidic layer as described herein, it is to be understood that it refers to a measure along a vertical axis. The term “outwardly” is intended to refer to a feature that extends towards an exterior side of a reference axis. The term “inwardly” is intended to refer to a feature that extends towards an interior side of a reference axis.

Various implementations of the cell culture device will now be described in greater detail.

### Cell Culture Device

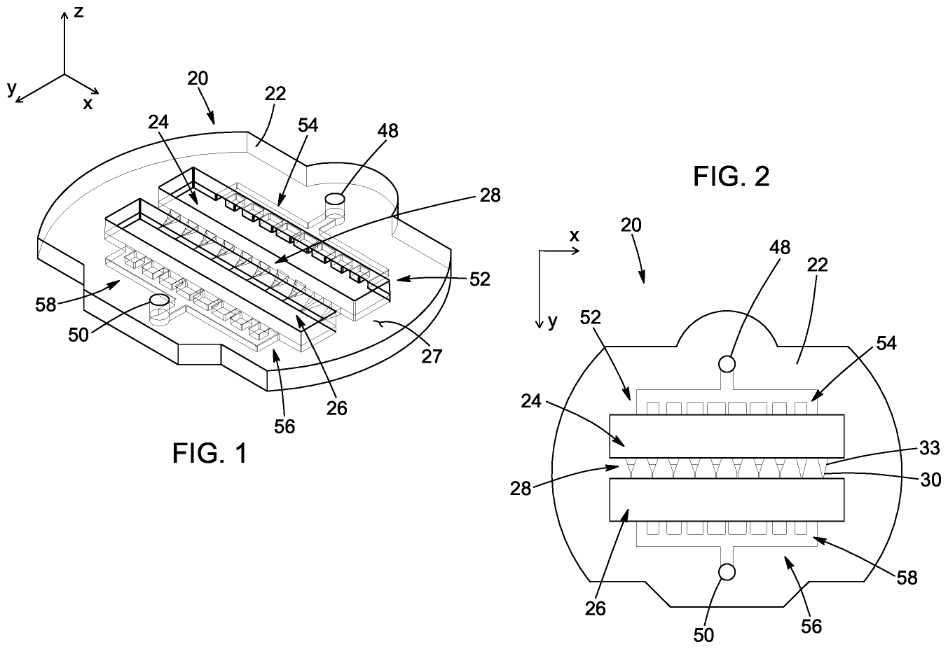

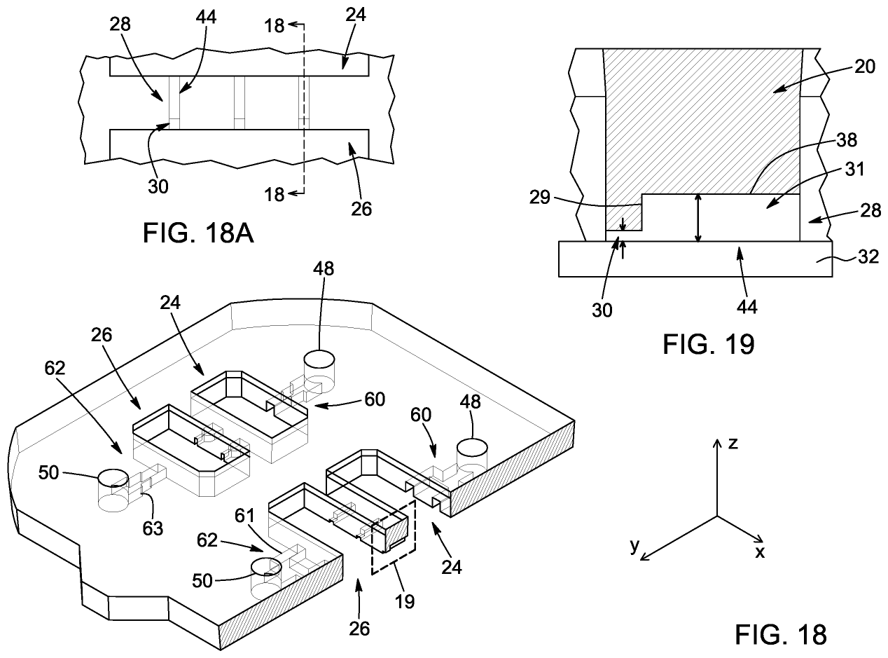

With reference to FIGS. 1-21, various implementations of a cell culture device 20 are shown. In each of the implementations shown, the cell culture device 20 includes a cell culture layer 22. The cell culture layer 22 includes an inlet reservoir 24 configured for receiving an inlet fluid medium therein, and an outlet reservoir 26 configured for receiving an outlet fluid medium therein. The cell culture layer 22 further includes a channel 28, or a plurality of channels 28, extending between the inlet reservoir 24 and the outlet reservoir 26 to establish fluid communication therebetween. In the illustrated implementations, the channel 28 is sized and configured for orienting the elongated component of a biological material toward the outlet reservoir 26.

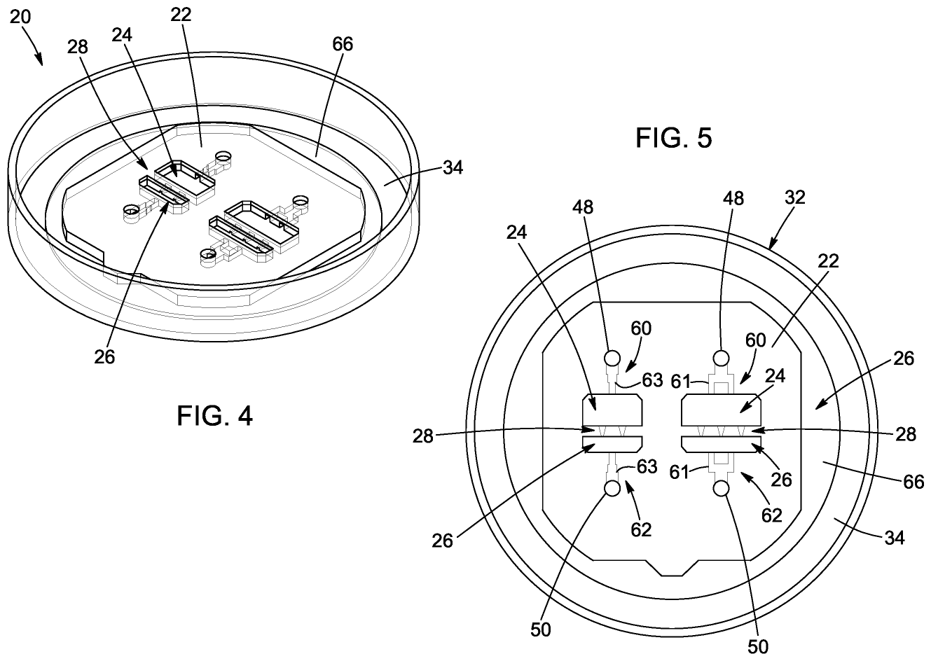

The cell culture device 20 can be configured to be inserted into a receptacle of a cell culture plate 32, e.g., into a cell culture dish, such as shown in FIGS. 4 and 5, for instance. In FIGS. 4 and 5, the cell culture plate 32 is exemplified as a petri dish. It is to be understood that the cell culture plate 32 can be a cell culture plate that includes a plurality of wells, and a cell culture layer 22 can be inserted into a corresponding well of the cell culture plate. Alternatively, the cell culture layer 22 can be integrated to, i.e., form an integral part of, the cell culture plate 32. When the cell culture layer 22 forms an integral part of the cell culture plate 32, the resulting device can be manufactured as a single unit.

Each of the components of the cell culture layer 22 will now be described in further detail in the paragraphs below.

### Cell Culture Layer

The cell culture layer 22 has a thickness defined between a top surface and a bottom surface thereof. In some implementations, the cell culture layer 22 can have a thickness ranging for instance from about 0.05 mm to about 100 mm. The cell culture layer 22 can be configured to be placed onto a cell culture layer-receiving surface of a cell culture plate 32. The cell culture layer 22 can be reversibly or irreversibly attached to the cell culture layer-receiving surface using any suitable method or technique, including but not limited to, compression, surface adhesion, ultrasonic welding, thermocompression bonding, plasma bonding, solvent-assisted bonding, laser-assisted bonding or adhesive bonding using glue or double adhesive tape.

Alternatively, and as mentioned above, the cell culture layer 22 can be integral to, e.g., moulded with, the cell culture plate 32.

The cell culture layer 22 can have various sizes and configurations. The size and configuration of the cell culture layer 22 can be adapted to the size and configuration of the receptacle or well of the cell culture plate 32 into which the cell culture layer 22 is intended to be inserted. For instance, in implementations where the cell culture layer 22 is intended to be inserted into a petri dish, the size of the cell culture layer 22 can be determined to fit within the receptacle formed by the petri dish, as shown in FIGS. 4 and 5. When the cell culture layer 22 is intended for insertion into a well of a multi-well cell culture plate, the size of the cell culture layer 22 can be adapted, e.g., reduced, so that the cell culture layer 22 can fit within a corresponding well of the multi-well cell culture plate.

The size and configuration of the cell culture layer 22 can also depend on its intended use, which can influence for instance the number of inlet reservoirs 24, the number of outlet reservoirs 26, and/or the number of channels 28, as well as the spatial distribution and size of each of these features of the cell culture layer 22.

The cell culture layer 22 can be made of any suitable polymeric material into which it is possible to carve, stamp or mold the one or more inlet reservoirs 24, the one or more outlet reservoirs 26, and the one or more channels 28. Examples of materials that can be suitable to produce the cell culture layer 22 can include, but are not limited to, polystyrene (PS), cyclo-olefin-copolymer (COC), cycloolefin polymer (COP), polymethyl methacrylate (PMMA), polycarbonate (PC), polyethylene (PE), polyethylene terephthalate (PET), polyamide (Nylon®), polypropylene (PP), polyether ether ketone (PEEK), Teflon®, polydimethylsiloxane (PDMS), and/or thermoplastic elastomer (TPE), as well as synthetic and biological materials such hydrogels, gelatin, collagen, chitosan, etc. In some implementations, the cell culture layer 22 can be made of a polymeric material that is transparent to light in order to facilitate optical analysis and visualization of the biological material and associated elongated component extending within the channels 28.

In some implementations, the cell culture layer 22 can be fabricated integral with the bottom wall 34 of the cell culture plate 32. Examples of materials that can be suitable to fabricate the cell culture layer 22 integral with the bottom wall 34 of the cell culture plate 32 can include, but are not limited to, polystyrene (PS), cyclo-olefin-copolymer (COC), cycloolefin polymer (COP), polymethyl methacrylate (PMMA), polycarbonate (PC), polyethylene (PE), polyethylene terephthalate (PET), polyamide (Nylon®), polypropylene or polyether ether ketone (PEEK), Teflon®, polydimethylsiloxane (PDMS), and/or thermoplastic elastomer (TPE), as well as synthetic and biological materials such hydrogels, gelatin, collagen, chitosan, etc.

### Inlet Reservoir and Outlet Reservoir

The cell culture layer 22 includes at least one inlet reservoir 24 and can include at least one outlet reservoir 26. In the implementation shown in FIGS. 1 and 2, the cell culture layer 22 includes a single inlet reservoir 24 and a single outlet reservoir 26, the single inlet reservoir 24 and the single outlet reservoir 26 forming a pair of reservoirs. In the implementation shown in FIGS. 4 and 5, the cell culture layer 24 includes two pair of reservoirs provided in a side-by-side relationship, each pair of reservoirs comprising an inlet reservoir 24 and an outlet reservoir 26. In alternative implementations, there can be more than one inlet reservoir associated with a single outlet reservoir, or there can be a single inlet reservoir associated with more than one outlet reservoir. It is to be understood that any combination of a number of inlet reservoirs and outlet reservoirs can be implemented, which can be determined for instance depending on the intended use of the cell culture layer 22.

The inlet reservoir 24 is configured to receive an inlet fluid therein. In some implementations, the inlet reservoir 24 can be further configured to receive a biological material, or a component of a biological material, therein. The inlet fluid can be a cell culture medium that enables survival and/or proliferation of the biological material or the component of the biological material received in the compartments of the cell culture layer 22, such as in the inlet reservoir 24 and/or in the channel 28. In some implementations, the inlet reservoir 24 is further configured to receive a test substance therein, such that the biological material or the component of the biological material present in the inlet reservoir and/or in the channel can be exposed to such test substance. As used herein, a test substance can be any type of substance that is desired to be tested to evaluate a response of a component of the biological material to that test substance. The test substance can take many forms, and can be for instance a liquid, a cream, a paste, an oil, a suspension, etc.

The outlet reservoir 26 can be configured to receive an outlet fluid therein. The outlet fluid can be the same as the inlet fluid or can be different from the inlet fluid. The outlet fluid can be a cell culture medium that enables survival and/or proliferation of the biological material or the component of the biological material received in the compartments of the cell culture layer 22, such as in the outlet reservoir 26 and/or in the channel 28. Alternatively, the outlet reservoir 26 can be configured to provide a substantially “dry” environment, such that the component of the biological material that eventually reaches the outlet reservoir 26 can be exposed to air. An outlet reservoir that enables exposing a component of the biological material to air can be suitable for instance when the biological material is hair. In such implementations, the hair follicle can be added to the inlet reservoir 24 or into a portion of channel 28 into which a cell culture medium is present, and the hair shaft can be inserted into the channel 28. The hair can then grow and can subsequently reach the outlet reservoir 26 with a portion of the hair being exposed to air.

In the implementations illustrated in FIGS. 1-21, the cell culture layer 22 is configured to extend substantially horizontally, with the inlet reservoir 24 and the outlet reservoir 26 being provided in a longitudinally spaced-apart relationship, i.e., along the y axis.

In some implementations, the outlet reservoir 26 is further configured to receive a test substance therein, such that the biological material present in the inlet reservoir is exposed to such test substance. As mentioned above, a test substance can be any type of substance that is desired to be tested to evaluate a response of the biological material to that test substance. The test substance can take many forms, and can be for instance a liquid, a cream, a paste, an oil, a suspension, etc.

The cell culture layer 22 can thus enable performing testing of substances in selected compartments of the cell culture layer 22 such as the inlet reservoir 24 and the outlet reservoir 26. The test substances can have different viscosities and/or solubilities depending on the compartment into which it is added. For instance, a cream can be added to one of the inlet reservoirs or the outlet reservoir, and a cell medium can be added to the remaining reservoir. Another example is an oil compound that can be added to one of the inlet reservoirs or the outlet reservoir, and a water-based compound that can be added to the remaining reservoir.

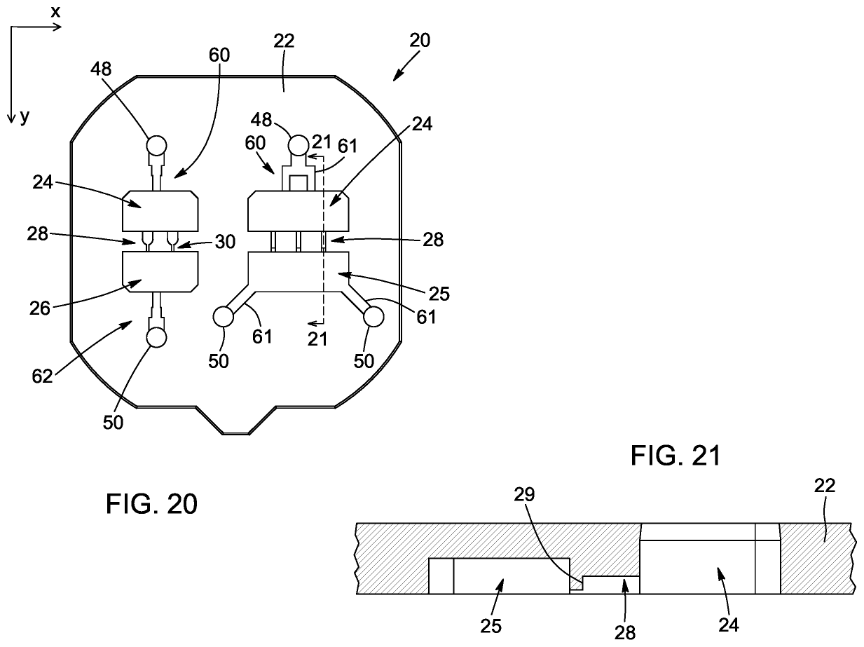

In some implementations, either one of the inlet reservoir 24 and the outlet reservoir 26 can be a closed reservoir, e.g., a reservoir that includes a top wall. FIG. 20 illustrates this type of implementation, with the outlet reservoir being configured as a closed outlet reservoir 25.

As the inlet reservoir 24 and the outlet reservoir 26 are in fluid communication with each other via the one or more channels 28, the inlet fluid and the outlet fluid can end up contacting each other at a certain location along a length of the cell culture layer 22, the length of the cell culture layer 22 being considered as extending along the y axis in the Figures. The meeting point of the inlet fluid and the outlet fluid can vary along the length of the cell culture layer 22, for instance depending on the configuration of the channels 28, and depending on whether the cell culture layer 22 is configured as a sloping cell culture layer. In particular, although referring to the cell culture layer 22 as extending substantially horizontally, the cell culture layer 22 can still be provided at a slight angle relative to the cell culture layer-receiving surface 34 of a cell culture plate 32. Thus, when referring to a sloping cell culture layer, it is meant that the cell culture layer 22 can be configured so as to be provided with a sloping angle with respect to the cell culture layer-receiving surface 34 of a cell culture plate 32. When considering the location of the inlet reservoir 24 relative to the outlet reservoir 26, the angle can be such that there is a descending slope from the inlet reservoir 24 to the outlet reservoir 26, or the angle can be such that there is an ascending slope from the inlet reservoir 24 to the outlet reservoir 26. For instance, when a cell culture medium is provided in the inlet reservoir 24 and in the outlet reservoir 26, and a test substance is placed into the outlet reservoir 26 but not in the inlet reservoir 24, it can be beneficial to provide the cell culture layer 22 with a slight descending slope toward the outlet reservoir 26 if it is desired to reduce the flow of the test substance into the inlet reservoir 24. Similarly, when a cell culture medium is provided in the inlet reservoir 24 and in the outlet reservoir 26, and a test substance is placed into the inlet reservoir 24 but not in the outlet reservoir 26, it can be beneficial to provide the cell culture layer 22 with a slight descending slope toward the inlet reservoir 24 if it is desired to reduce the flow of the test substance into the outlet reservoir 26.

In some implementations, still when the inlet reservoir 24 and the outlet reservoir 26 are provided in a longitudinally spaced-apart relationship, one of the compartments of the cell culture layer 22 can include an anti-return feature. An anti-return feature refers to a structural feature that encourages the flow of a fluid in a preferential direction. For instance, when a cell culture medium is provided in the inlet reservoir 24 and in the outlet reservoir 26, and a test substance is inserted into the outlet reservoir 26 but not in the inlet reservoir 24, it can be beneficial to provide an anti-return feature in proximity of the outlet reservoir 26, for instance in proximity of a transition between the channel 28 and the outlet reservoir 26, to reduce backflow of the test substance into the inlet reservoir 24 while maintaining fluid communication between the inlet reservoir 24 and the outlet reservoir 26. Similarly, when a cell culture medium is provided in the inlet reservoir 24 and in the outlet reservoir 26, and a test substance is inserted into the inlet reservoir 24 but not in the outlet reservoir 26, it can be beneficial to provide an anti-return feature in proximity of the inlet reservoir 24, for instance in proximity of a transition between the channel 28 and the inlet reservoir 24, to reduce flow of the test substance into the outlet reservoir 26 while maintaining fluid communication between the inlet reservoir 24 and the outlet reservoir 26.

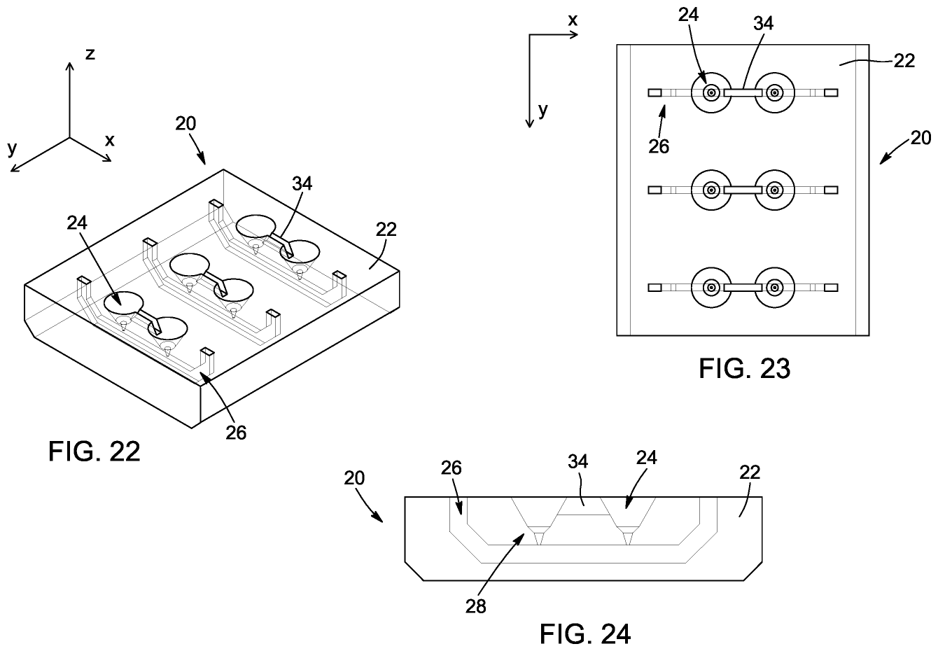

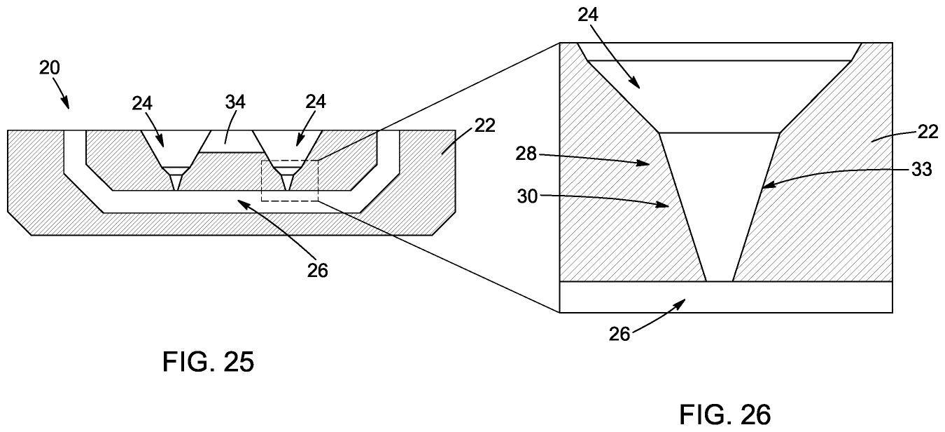

Referring to FIGS. 22-26, in this implementation of the cell culture layer 22, the cell culture layer 22 is also configured to extend substantially horizontally, although the inlet reservoir 24 and the outlet reservoir 26 are provided in a superposed relationship across the thickness of the cell culture layer 22, such that the outlet reservoir 26 is positioned underneath the inlet reservoir 24. In such implementations, gravity will influence the flow of the cell culture media from the inlet reservoir 24 toward the outlet reservoir 26. In the implementation shown, the outlet reservoir 26 is shaped as a U, i.e., is U-shaped, and each of the branches of the U opens up at the level of the top surface 27 of the cell culture layer 22. Although in the implementation shown, both of the branches of the U open up at the level of the top surface 27 of the cell culture layer 22, it is to be understood that only one of the branches can open up at the level of the top surface 27 of the cell culture layer 22. This opening enables a cell culture media or a test substance to be added to the outlet reservoir 26. In this implementation, the inlet reservoir 24 is an open-top inlet reservoir to enable addition of a cell culture medium or of a test substance therein. In some implementations and as illustrated in FIGS. 22-26, the inlet reservoir 24 can include a downwardly converging portion that converges toward the channel 28 and thus toward the outlet reservoir 26. The downwardly converging portion of the inlet reservoir 24 can facilitate addition of a cell culture medium and/or test substance therein.

Still referring to FIGS. 22-26, adjacent inlet reservoirs 24 can be placed in fluid communication via an inlet reservoir bridge 34 that extends therebetween. In some implementations, the inlet reservoir bridge 34 can be used for the addition of the cell culture medium and/or the test substance that eventually flows into the inlet reservoirs 24, which can contribute to reduce the turbulence of the fluid added and the impact of this turbulence on the stability of the biological material or the component of the biological material that is received in the inlet reservoirs 24.

The configuration of the inlet reservoir 24 or the outlet reservoir 26 can be chosen according to the type of biological material that is intended to be received therein. For instance, an inlet reservoir 24 that is configured to receive elongating organoids such as neurospheres, neuro-organoids, or any other type of biological material containing neurons, therein can be chosen to include the downwardly converging portion as described above. In such implementations, the downwardly converging portion of the inlet reservoir 24 can contribute to stabilize the neurospheres or the neuro-organoids against the converging walls of the inlet reservoir 24 and to direct axons toward the channel 28.

In some implementations, the inlet reservoir 24 and/or the outlet reservoir 26 can have a depth, along the z-axis, ranging from about 0.1 mm to about 50 mm. In some implementations, the inlet reservoir 24 and/or the outlet reservoir 26 can have a width, along the x-axis, ranging from between about 0.1 mm to about 200 mm. The width of the inlet reservoir 24 and the outlet reservoir 26 can vary depending on the number of channels 28 that extends therebetween, among other factors. In some implementations, the inlet reservoir 24 and/or the outlet reservoir 26 can have a length, along the y-axis, ranging from between about 0.1 mm to about 200 mm. It is to be noted that the dimensions given above are examples only, and that various sizes are also possible, for instance depending on the intended use of the cell culture device 20.

### Channels

The inlet reservoir 24 and the outlet reservoir 26 are in fluid communication via at least one channel 28 extending therebetween. The number of channels 28, or the density of the channels 28, between the inlet reservoir(s) 24 and outlet reservoir(s) 26 can vary. For instance, in some implementations, there can be anywhere from 1 to 100 channels between a pair of an inlet reservoir 24 and an outlet reservoir 26, or there can be more than 100 channels between a pair of an inlet reservoir 24 and an outlet reservoir 26. The distribution of the channels 28 can vary along the width of the cell culture layer 22 and thus of the inlet reservoir 24 or outlet reservoir 26, the width of the cell culture layer 22 being considered as extending along the x-axis in the Figures. For instance, the channels 28 can be provided at a distance between each other that is relatively constant, such as illustrated in FIGS. 1 and 2. In other implementations, the channels 28 can be provided as groups of channels and be spatially distributed at various distances from each other along the width of the cell culture layer 22. In some implementations, the channels 28 of a given cell culture layer 22 can be substantially similar to each other. Alternatively, the cell culture layer 22 can include a succession of channels 28 that are configured differently from each other, can include groups of channels 28 that are configured substantially similar to each other within a same group, with the channels 28 of each group being different from each other.

The channels 28 of the cell culture layer 22 are configured to at least enable orienting an elongated component of a biological material away from the inlet reservoir 24. As used herein, the expression “elongated component” of a biological material refers to any type of biological structure or cell type that typically grows in an elongated fashion, such as hairs or axons, for instance. The channels 28 described herein provide a stabilizing environment for such elongated component of a biological material to grow or to be maintained in an elongated configuration, which can contribute to preventing entanglement and promote a directional growth of the elongated component.

In some implementations, the channel 28 can be configured with dimensions and a structure that enables maintaining the biological material or a given component of the biological material in a given position, which can enable avoiding or reducing the movement of the biological material or the given component of the biological material along the x-axis, the y-axis and/or the z-axis. Maintaining a given component of the biological material or the biological material in a given position can also contribute to avoiding or reducing floating of the biological material in the liquid medium and keeping it immersed in a specific solution or medium, and/or avoiding or reducing movement of the biological material or of a component of the biological material from one compartment to another, and/or avoiding or reducing movement of the biological material towards one or the other reservoir, and or to the sides. The channel 28 can also contain an additional structure to lock the biological material or a component of the biological material in place to orient the direction of the biological material or the component of the biological material from one reservoir or another. For example, the follicles side can be facing one reservoir while the hair coming out of the follicles can be facing another reservoir.

In some implementations, the channel 28 can include, or can consist of, an elongated component receiving portion 30 that is sized and configured for orienting the elongated component of the biological material away from the inlet reservoir 24. For instance, in some implementations when the multicellular component of the biological material is received in the inlet reservoir 24, the channel 28 can include an elongated component receiving portion 30 that is sized and configured for selectively retaining the multicellular component of the biological material upstream of the channel 28, i.e., within the inlet reservoir 24, while the elongated component extends within the channel 28, i.e., within the elongated component receiving portion 30. The elongated component receiving portion 30 can also be configured to constrain or hold the elongated component of the biological material in position, i.e., to reduce the movement of the elongated component of the biological material. For instance, when the cell culture layer 22 is configured to culture hairs and associated follicles, the elongated component receiving portion 30 can be sized and configured to hold the hair in place and prevent it from passing through the channel 28 completely, as hair follicles are thicker at the follicle end and thinner at the hair end.

In some implementations, the channel 28 can further include a multicellular component receiving portion 31 configured for receiving at least a portion of the multicellular component of the biological material therein. When the channel 28 includes a multicellular component receiving portion 31, the elongated component receiving portion 30 is provided downstream of the multicellular component receiving portion 31. In implementations where the multicellular component of the biological material is received in the inlet reservoir 24, the multicellular component receiving portion can be omitted. In some implementations, a portion of the multicellular component of the biological material can be received in the inlet reservoir 24, and another portion of the multicellular component of the biological material can be received in the multicellular component receiving portion 31. When the channel 28 includes a multicellular component receiving portion 31, with an elongated component receiving portion 30 provided downstream of the multicellular component receiving portion 31, the elongated component receiving portion 30 can be sized and configured for orienting the elongated component of the biological material toward the outlet reservoir and for preventing the multicellular component from travelling past the multicellular component receiving portion 31.

It is to be understood that the size and configuration of the channel 28, or of portions of the channel 28, can be adapted to selectively retain a multicellular component of the biological material therein and preventing the multicellular component to travel downwardly past the multicellular component receiving portion 31, and to maintain the elongated component of the biological material in an elongated configuration downstream of the multicellular component receiving portion 31, i.e., in the elongated component receiving portion 30.

One of the objectives of the interaction of the channels 28 with the inlet reservoir 24 is to enable the elongated component of the biological material to be cultured in an organized fashion while another component of the biological material, or another biological material, remains in the inlet reservoir 24 of the cell culture layer 22 or in the multicellular component receiving portion 31 of the channel 28, so any configuration of the channels 28 that can achieve such objective can be suitable.

For instance, when the cell culture layer 22 is configured to culture neurons, the inlet reservoir 24 or the multicellular component receiving portion 31 of the channel 28 can be configured to receive clusters of cell bodies therein, or neurospheroids, neuro-organoids, or any other type of biological material containing neurons, and the channel 28, or the remainder of the channel if the channel includes a multicellular component receiving portion 31, can be configured to receive the axons, the channel 28 comprising an elongated component receiving portion 30 that is sized and configured to maintain the elongated configuration of the axons. Examples of types of neuronal cells that can be used for the preparation of the compartmentalized in vitro model can include mammalian neurons, such as rodent embryonic neurons, and neurons derived from induced pluripotent stem cells, such as human induced pluripotent stem cells, for instance. The option of growing different types of neuronal cells when preparing the compartmentalized in vitro model can increase the versatility of the resulting biological model, which in turn can offer a wider range of opportunities for the various needs of the industry. Using human-derived cells can also be beneficial to provide reproducible and accurate results that can facilitate the translation of the drugs or compounds testing to human studies.

In another example, the cell culture layer 22 can be configured to culture hairs and associated follicles. In such implementations, the inlet reservoir 24 or the multicellular component receiving portion 31 of the channel 28 can be configured to culture the follicles, while the channel 28, or the remainder of the channel 28 if the channel includes a multicellular component receiving portion 31, can be configured to receive the hairs, the channel 28 comprising an elongated component receiving portion 30 that is sized and configured to maintain the elongated configuration of the hairs. In this example, one hair can be received in a given channel 28 of the cell culture layer 22, or alternatively, each channel 28 of the cell culture layer 22 can be configured to receive more than one hair.

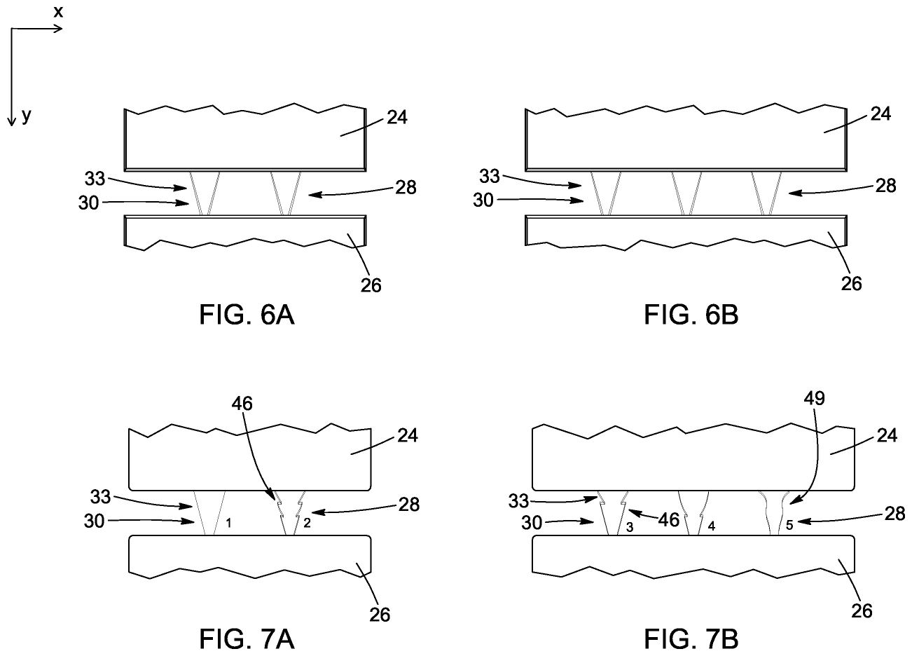

In some implementations, the channel 28 includes a converging portion 33 that converges away (tapers inwardly such as shown in FIGS. 6A and 6B for instance) from the inlet reservoir 24, i.e., toward the outlet reservoir 26, that can contribute to orienting the elongated component of the biological material toward the outlet reservoir 26.

In implementations where the channel 28 includes a multicellular component receiving portion 31, the converging portion 33, if present, can be provided downstream of the multicellular component receiving portion 31. In such implementations, the converging portion 33 can define at least a portion of the elongated component receiving portion 30. Alternatively, at least a portion of the multicellular component receiving portion 31 can form part of the converging portion 33, or be defined by the converging portion 33, of the channel 28. In other words, at least a portion of the multicellular component receiving portion 31 can be defined by a portion of the channel 28 that is converging inwardly toward the outlet reservoir 26 and that is sized and configured to receive a multicellular component of a biological material therein, while the remainder of the converging portion 33 is sized and configured to receive an elongated component of the biological material therein. Accordingly, when the channel 28 comprises a converging portion 33, the converging portion 33 can define at least a portion of the multicellular component receiving portion 31, or the converging portion 33 can define at least a portion of the elongated component receiving portion 30. In some implementations, the converging portion 33 can define both at least a portion of the multicellular component receiving portion 31 and at least a portion of the elongated component receiving portion 30.

In the implementations shown in FIGS. 1-21, i.e., when the inlet reservoir 24 and the outlet reservoir 26 are provided in a longitudinally spaced-apart relationship (i.e., spaced-apart along the y-axis), the channel 28 can be considered to include side walls 36, a top wall 38, and optionally, a back wall (not shown). In the implementations shown in FIGS. 1-21, the back wall is not defined in the cell culture layer 22 itself, but the bottom wall 34 of the cell culture plate 32 (or cell culture layer-receiving surface) provides the confinement of the channel 28 along this plane.

In the implementations shown in FIGS. 22-26, i.e., when the inlet reservoir 24 and the outlet reservoir 26 are provided in a superposed relationship, the channel 28 can be considered to include a peripheral wall, or side walls.

The converging portion 33 of the channel 28 can take various configurations. In the implementations shown in FIGS. 1-21, the converging portion 33 converges toward the outlet reservoir 26 along the longitudinal axis of the cell culture layer 22, i.e., along the y-axis of the cell culture layer 22. To achieve the convergence toward the outlet reservoir 26, the converging portion 33 can have a converging wall along the x-axis or the z-axis, or along both the x-axis and the z-axis.

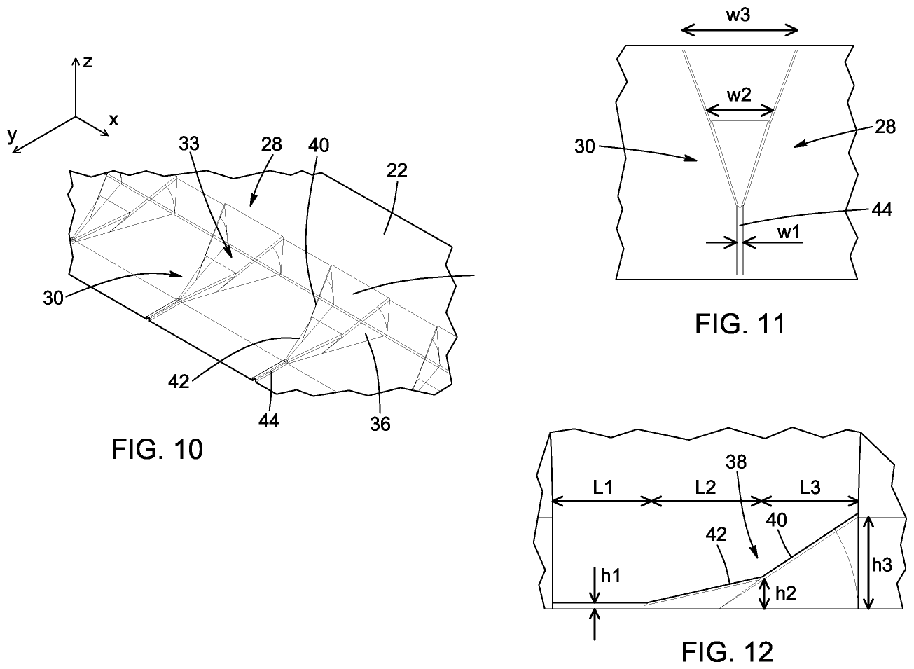

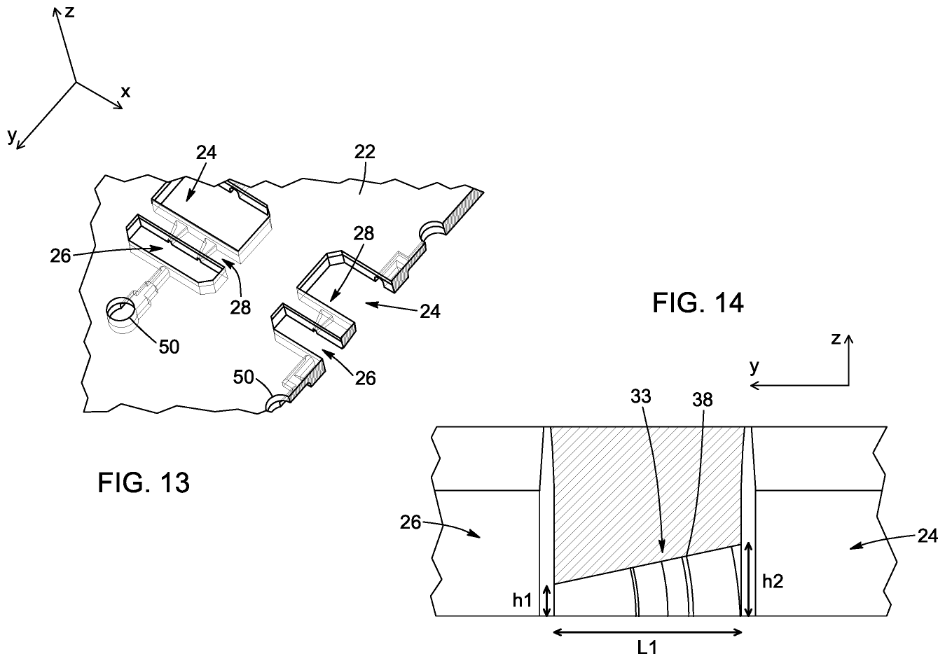

In the examples shown in FIGS. 1-14, the converging portion 33 of the channel 28 converges inwardly toward the outlet reservoir 26 along the y-axis of the cell culture layer 22 via a converging wall along both the x-axis and the z-axis. Thus, in order to provide the channel 28 with a converging portion 33 having a converging wall along both the x-axis and the z-axis, at least one of the top wall 38 and the back wall, if present, can be a converging wall, and at least one of the side walls 36 can be a converging wall. It is to be noted that although the converging walls along both the x-axis and the z-axis illustrated in FIGS. 1-14 are shown either as a succession of converging walls (for instance as shown in FIGS. 10-12) or a continuous converging wall (for instance as shown in FIGS. 13-14), the convergence of the converging wall can be the result of a step change, such as shown in FIG. 19 for example (in FIG. 19, the step change is along the z-axis), or can be provided as a curved converging wall.

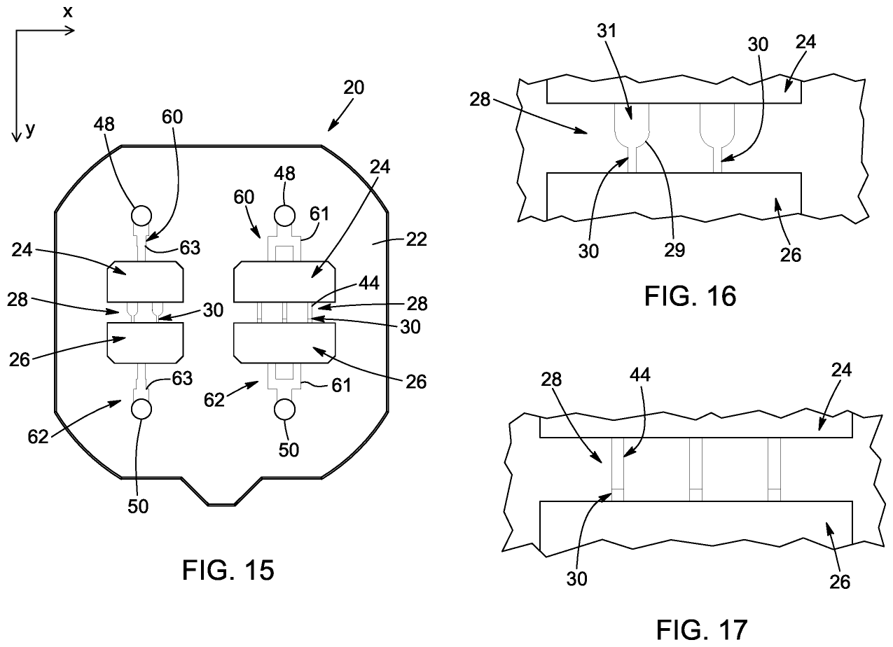

In the example shown in FIGS. 15-21, for channels 3, 4 and 5, the converging portion 33 of the channel 28 converges toward the outlet reservoir 26 along the y-axis of the cell culture layer 22 via a converging wall that converges along the z-axis. In such implementations, the converging portion 33 of the channel 28 can be provided by the converging of at least one of the top wall 38 and the back wall, if present, along the z-axis. In FIGS. 15-21, for channels 3, 4 and 5, the converging wall along the z-axis includes a step change, in contrast to a succession of converging walls or a continuous converging wall, although a succession of converging walls or a continuous converging wall can be present instead of the step change.

In the example shown in FIGS. 15-21, for channels 1 and 2, the converging portion 33 of the channel 28 converges toward the outlet reservoir 26 along the y-axis of the cell culture layer 22 via a converging wall that converges along the x-axis only. In such implementations, the converging portion 33 of the channel 28 can be provided by the converging of at least one side walls along the x-axis.

In some implementations, the converging portion 33 of the channel 28 can be shaped as a cup, the curved surface of the cup forming the converging portion 33 of the channel 28. In such implementations, the curved surface of the cup forming the converging portion 33 can be sized and configured such that a multicellular component of the biological material lightly leans against the curved surface of the cup, to prevent the multicellular component from travelling downstream toward the outlet reservoir 26. Furthermore, in such implementations, the converging portion 33 can be considered as including a multicellular component receiving portion 31 that receives the multicellular component therein, with the remainder of the channel 28, i.e., the elongated component receiving portion 30, is sized and configured to receive an elongated component of the biological material therein.

In some implementations, the converging portion 33 can include a frustoconical converging portion, similar to the example shown in FIGS. 22-26 for instance. In some implementations, the converging portion 33 can include a frustopyramidal converging portion, similar to the example shown in FIG. 10 for instance.

In some implementations, the converging portion 33 of the channel 28 can include converging sections that are each converges at a corresponding angle toward the outlet reservoir 26. FIG. 12 illustrates an example of a converging portion 33 that includes a first converging section 40 and a second converging section 42, the second converging portion 42 being provided downstream of the first converging section 40. The first converging portion 40 and the second converging portion 42 are thus provided successively from the inlet reservoir 24 to the outlet reservoir 26. In some implementations and as illustrated in FIGS. 1, and 8-12, the first converging section 40 converges toward the outlet reservoir 26 at an angle that is larger than the angle at which the second converging section 42 converges toward the outlet reservoir 26. In some implementations, the larger angle of the first converging section 40 can facilitate capturing and orienting the elongated component of the biological material within the channel 28 and toward the outlet reservoir 26. In some implementations, a first converging portion 40 that has a larger angle compared to the subsequent converging portion can enable forming a multicellular component receiving portion 31 that is sized and configured from receiving a multicellular component of a biological material therein. The smaller angle of the second converging section 42 can then contribute to further stabilize the elongated component. The extent of variation between the angle of the first converging section 40 and the second converging section 42 can vary, for instance depending on the application and the stabilizing effect sought-after. In some implementations, more than two converging sections can be present, to gradually achieve a desired overall converging portion.

In some implementations, the channel 28 can have substantially constant width along the x-axis, or can include a constant width portion 44 provided along at least a portion located downstream of the converging portion 33, the constant width portion 44 having a substantially constant width throughout its length, as illustrated for instance in FIGS. 10, 11, 16 and 17. It is to be understood that the constant width portion 44 of the channel 28 can be a rectangular prism having a substantially constant width along the x-axis of the cell culture device 20, or a tubular portion having a substantially constant diameter along the x-axis of the cell culture device 20. The presence of the constant width portion 44 can further contribute to stabilizing the elongated component of the biological material. The width or diameter of the constant width portion 44 can vary depending on the application of the cell culture device 20, and can be determined for instance in accordance with the type of biological material that is intended to be inserted and cultured in the channel 28. For example, the diameter, or width, of the constant width portion 44 can be determined so as to obtain a given ratio between the width of the constant width portion 44 and the width of the elongated component.

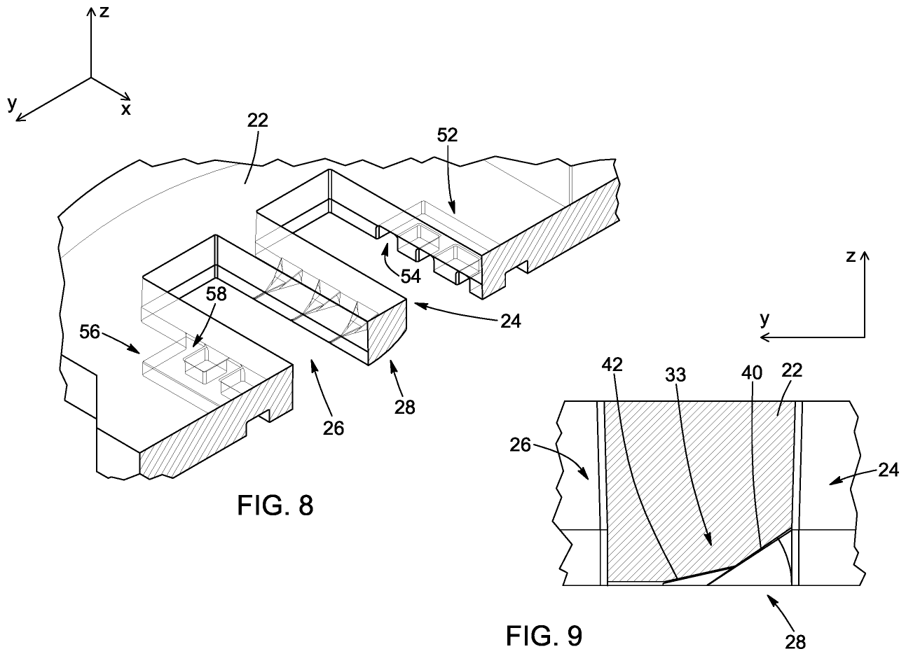

In some implementations, the transition between successive converging sections of the converging portion 33 can include a curved transition. Alternatively, the transition between successive converging sections of the converging portion 33 can include a sharp transition. For instance, in the implementations, shown in FIGS. 8-12, the transition between the first converging portion 40 and the second converging portion 42 can be considered a sharp transition, i.e., a transition that is not curved. In some implementations, the presence of a curved transition or of a sharp transition between a first converging portion and a second converging portion can also contribute to stabilizing the elongated component of the biological material given the formation of internal edges that results from the transition.

Referring now more particularly to FIGS. 7A and 7B, there are shown various examples of types of channels that can be suitable for a cell culture layer 22 as described herein. In FIGS. 7A and 7B, channel 1 illustrates an implementation of a channel 28 that includes a converging portion 33 that converges from the inlet reservoir 24 to the outlet reservoir 26 substantially continuously along at least the x-axis. Channel 2 illustrates an implementation of a channel 28 that includes a converging portion 33 that converges from the inlet reservoir 24 to the outlet reservoir 26, and that includes a series of inwardly protruding members 46 that defines arrowhead features converging toward the outlet reservoir 26. The number of inwardly protruding members 46 and their location along the y-axis can vary, as shown in instance in channels 3 and 4. In some implementations, the converging portion 33 can include a neck portion 49 that can also contribute to stabilizing the elongated component of the biological material, such as shown in channel 5. It is to be understood that the various features of the channels 28 shown in FIGS. 7A and 7B can be combined in various ways to achieve a desired channel for an intended application of the cell culture layer 22. For instance, a channel 28 can include a combination of one or more inwardly protruding members 46 and a neck portion 49, with the neck portion 49 being located either upstream or downstream of the one or more inwardly protruding members 46.

In some implementations, and as mentioned above, the channel 28 can include a multicellular component receiving portion 31 that is sized and configured to retain a multicellular component of a biological material therein, which can also be referred to as a non-elongated component of a biological material, upstream of the elongated component receiving portion 30. In other words, the reduction in size of the channel 28 from the multicellular component receiving portion 31 to the elongated component receiving portion 30 can be such that at a certain location along the length, i.e., along the y-axis, of the channel 28, the channel 28 becomes too small for the multicellular component of the biological material or the non-elongated component of the biological material to travel further down along the y-axis of the channel 28. In such implementations, the channel 28 thus includes at least one section that corresponds to the elongated component receiving portion 30 and that has a smaller width or diameter than the width or diameter of the multicellular component of the biological material that will be received upstream of the elongated component receiving portion 30.

For instance, when the biological material is a neurosphere, the cluster of neuronal cell bodies of the neurosphere can be received into a multicellular component receiving portion 31 of a channel 28, and the axons can extend in an elongated component receiving portion 30 of the channel 28 that is sized according to the size of the neurosphere, such that the neurosphere containing the cell bodies is retained upstream of the elongated component receiving portion 30. For example, and with reference to FIG. 19, the cluster of neuronal cell bodies of the neurosphere can be retained in the wider part of the channel 28, which would be considered as a multicellular component receiving portion 31, and can rest on the inwardly converging wall 29 that converges inwardly toward the center of the channel 28, while the axons extend downstream in the elongated component receiving portion 30.

When the multicellular component receiving portion 31 is shaped as a cup, the size and configuration of the multicellular component receiving portion 31 can be adapted in accordance with the shape of the multicellular component of the biological material or the non-elongated component of the biological material, and can substantially match the shape of the multicellular component of the biological material or the non-elongated component of the biological material. For instance, for a neurosphere that is substantially circular, the multicellular component receiving portion 31 can be sized such that the cluster of neuronal cell bodies of the neurosphere leans lightly against the inwardly converging wall 29 of the converging portion 33, with the axons extending within the elongated component receiving portion 30 provided downstream of the multicellular component receiving portion 31, or downstream of the inwardly converging wall 29.

These configurations of the channel 28 can also be implemented for instance when a first biological material is a follicle, and a second biological material is a hair as an elongated component. In such implementation, the multicellular component receiving portion 31 can be sized and configured such that the follicle is retained within the multicellular component receiving portion 31, with the hair extending further into the channel 28 downstream of the multicellular component receiving portion 31. In this example, the follicle would thus be considered as a multicellular component of a biological material (or non-elongated), and the hair shaft would be considered an elongated component of the biological material. A hair shaft can typically have a diameter of 50 microns to 150 microns, and a follicle can typically have a diameter of about 200 microns, such that an elongated component receiving portion 30 having at least a portion that has a diameter of less than 200 microns can enable retaining the hair shaft therein and maintaining it in an elongated configuration, while the follicle is retained upstream in the multicellular component receiving portion 31. This type of configuration of the channel 28 can prevent the hair shaft from travelling down the channel 28, as the follicle retains it via its interaction with the multicellular component receiving portion 31.

When the converging portion 33 includes a succession of converging walls or a continuous converging wall, at least a section of the converging portion 33 can be sized as a multicellular component receiving portion 31 to retain a multicellular component of a biological material or a non-elongated component of a biological material upstream of the elongated component receiving portion 30 of the channel 28.

It is to be noted that although the configurations of the channels 28 shown in FIGS. 1-21 are illustrated for a cell culture layer 22 that includes an inlet reservoir 24 and an outlet reservoir 26 that are provided according to a longitudinally spaced-apart relationship, it is to be understood that the described configurations can also be implemented when the inlet reservoir 24 and the outlet reservoir 26 are provided according to a superposed relationship, such as shown in FIGS. 22-26.

In some implementations, the channel 28 can have a substantially constant width, or diameter, along its length, i.e., between the inlet reservoir 24 and the outlet reservoir 26. In such implementations, the channel 28 can be sized for orienting the elongated component of the biological material toward the outlet reservoir 26 and for contributing to maintaining the elongated component of the biological material in position, or stabilized, during culture. For instance, a channel 28 having a substantially constant width between the inlet reservoir 24 and the outlet reservoir 26 can be implemented when the biological material itself has a converging shape, or when the multicellular component of the biological material has a larger size compared to the size of the elongated component of the biological material. An example of such biological material is a hair shaft and associated follicle. In this example, the follicle would thus be considered as a multicellular component of a biological material (or non-elongated), and the hair shaft would be considered an elongated component of the biological material. In such implementations, the channel 28 can have a sufficiently small width to prevent the follicle from entering into the channel 28, such that the follicle remains in the inlet reservoir 24 and the hair grows into the channel 28. For instance, as a hair shaft can typically have a diameter of 50 microns to 150 microns, and a follicle can typically have a diameter of about 200 microns, the channel 28 can have a constant width of less than 200 microns to enable retaining the hair shaft therein and maintaining it in an elongated configuration, while the follicle is retained in the inlet reservoir 24. This type of configuration of the channel 28 can prevent the hair shaft from travelling down into the channel 28. Other examples of biological material having a converging shape also include neurospheres or neuro-organoids. In such implementations, the channel 28 can be sized according to the size of the neurosphere, i.e., such that the cluster of cells of the neurosphere forming the multicellular component of the biological material can be retained in the inlet reservoir 24, and the axons can extend within corresponding channels as the elongated component of the biological material.

The channels 28 of the cell culture layer 22 can be carved, stamped or molded into the cell culture layer 24. In some implementations, the cell culture layer 22 includes channels 28 that are open-top, i.e., that are open to the atmosphere unless a cover is deposited onto the cell culture layer 22. In such implementations, it is thus meant that the top wall 38 of the channels 28 as described above is omitted. When the top wall 38 of the channels 28 is omitted, the converging portion 33 of the channels 28 can be achieved by the side walls 40 converging along the x-axis. As mentioned above, the channels 28 can include a back wall, or the bottom wall 34 of the cell culture plate 32 can act as a back wall, or an additional layer or membrane can be provided underneath the bottom surface of the cell culture layer 22, i.e., between the top surface of the bottom wall 34 of the cell culture plate 32 and the cell culture layer 22. The membrane can include for instance a biological material, or can be made of a biologically inert material.

With reference to FIGS. 11, 12 and 14, Table 1 below provides examples of dimensions that a channel 28 of a cell culture layer 22 can have. It is to be understood that the dimensions given in Table 1 are examples only, and that multiple variations of these dimensions are possible. The inlet reservoir 24 and the outlet reservoir 26 shown in FIGS. 1 and 2 can have a length along the y-axis that ranges for instance from 5 mm to 20 mm, for example. The inlet reservoir 24 and the outlet reservoir 26 shown in FIGS. 1 and 2 can have a width along the x-axis that ranges for instance from 20 mm to 70 mm, for example. Of course, these dimensions can vary greatly, for instance for a configuration of the cell culture layer 22 illustrated in FIGS. 4 and 5, where a plurality of inlet reservoirs 24 and a plurality of outlet reservoirs 26 are present. As mentioned above, the size and number of inlet reservoirs and outlet reservoirs can vary depending on the cell culture plate with which it is intended to be used, and depending on the biological material it is intended to receive.

TABLE 1

| h1 (mm) | h2 (mm) | h3 (mm) |
|---|---|---|
| 0.05-0.3 | 0.2-1 | 0.5-4 |
| L1 (mm) | L2 (mm) | L3 (mm) |
| 0.5-3 | 1-3 | 0.5-3 |
| w1 (mm) | w2 (mm) | w3 (mm) |
| 0.05-0.4 | 0.5-3 | 1-4 |

### Additional Optional Features of the Cell Culture Layer

In some implementations, the cell culture layer 22 can further include an inlet well 48 in fluid communication with the inlet reservoir 24, and/or an outlet well 50 in fluid communication with the outlet reservoir 26. The inlet well 48 is configured to receive an inlet fluid medium therein, and the outlet well 50 is configured to receive an outlet fluid medium therein, which can be the same or different than the inlet fluid medium.

The fluid communication between the inlet well 48 and the inlet reservoir 24, or between the outlet well 50 and the outlet reservoir 26, can be established in various ways. In the implementation illustrated in FIGS. 1 and 2, the fluid communication between the inlet well 48 and the inlet reservoir 24 can be established via an inlet manifold 52 extending between the inlet well 48 and the inlet reservoir 24. The inlet manifold 52 includes a plurality of inlet reservoir channels 54. The inlet manifold 52 and the plurality of inlet reservoir channels 54 are configured to facilitate directing the flow of the inlet fluid medium from the inlet well 48 to the inlet reservoir 24 with reduced turbulence. The width, along the x-axis, of the inlet reservoir channels 54 can vary along the width of the cell culture layer 22, as illustrated in FIGS. 1 and 2. FIGS. 1 and 2 illustrate inlet reservoir channels 54 that have a width that is smaller in a center region of the inlet manifold 52 compared to the width of the inlet reservoir channels 54 in outer regions of the inlet manifold 52. It is to be understood that the number of inlet reservoir channels 54, their width and their length can vary, for instance depending on the intended application of the cell culture layer 22, and that FIGS. 1 and 2 are given for illustrative purposes only.

Similarly, still in the implementation illustrated in FIGS. 1 and 2, the fluid communication between the outlet well 50 and the outlet reservoir 26 can be established via an outlet manifold 56 extending between the outlet well 50 and the outlet reservoir 26. The outlet manifold 56 includes a plurality of outlet reservoir channels 58. The outlet manifold 56 and the plurality of outlet reservoir channels 58 are configured to facilitate directing the flow of the inlet fluid medium from the outlet well 50 to the outlet reservoir 26 with reduced turbulence. The width, along the x-axis, of the outlet reservoir channels 58 can vary along the width of the cell culture layer 22, as illustrated in FIGS. 1 and 2. FIGS. 1 and 2 illustrate outlet reservoir channels 58 that have a width that is smaller in a center region of the outlet manifold 56 compared to the width of the outlet reservoir channels 58 in outer regions of the outlet manifold 56. It is to be understood that the number of outlet reservoir channels 58, their width and their length can vary, for instance depending on the intended application of the cell culture layer 22, and that FIGS. 1 and 2 are given for illustrative purposes only. Moreover, although the inlet reservoir channels 54 and the outlet reservoir channels 58 are illustrated as being similar to each other in FIGS. 1 and 2, it is to be understood that the configuration of the inlet reservoir channels 54 and the outlet reservoir channels 58 can be different from each other, within a same cell culture layer 22.

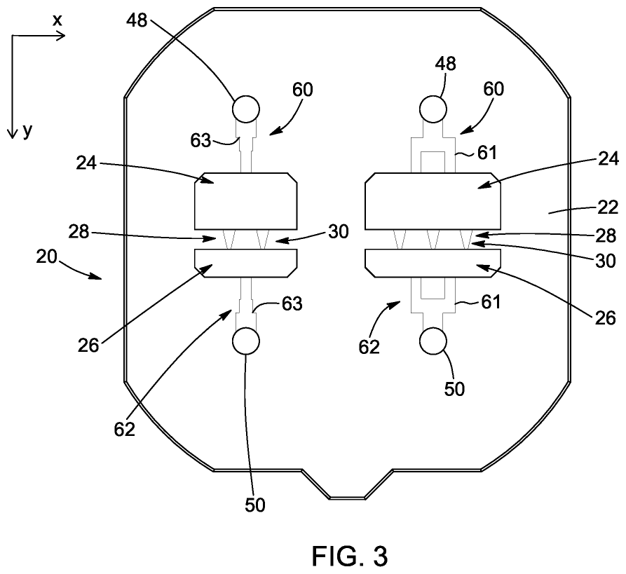

Turning to FIG. 3, fluid communication between the inlet well 48 and the inlet reservoir 24 can be established via an inlet reservoir channel 60 extending between the inlet well 48 and the inlet reservoir 24, the inlet reservoir channel 60 being configured for directing flow of the inlet fluid medium into the inlet reservoir 24 with reduced turbulence. In some implementations, the inlet reservoir channel 60 is a converging inlet reservoir channel that converges toward the inlet reservoir 24, as illustrated in FIG. 3, on the left-hand side of the cell culture layer 22. The converging inlet reservoir channel can converge substantially continuously toward the inlet reservoir 24, or the converging inlet reservoir channel can include step changes 63 that overall result in the converging toward the inlet reservoir 24, as shown in FIG. 3. In addition, and as shown on the right-hand of the cell culture layer 22, the inlet reservoir channel 60 and/or the outlet reservoir channel 62 can include reservoir channels 61 to enable fluid communication with the inlet reservoir 24 and the outlet reservoir 26, respectively. Similarly, fluid communication between the outlet well 50 and the outlet reservoir 26 can be established via an outlet reservoir channel 62 extending between the outlet well 50 and the outlet reservoir 26, the outlet reservoir channel 62 being configured for directing flow of the inlet fluid medium into the outlet reservoir 26 with reduced turbulence. In some implementations, the outlet reservoir channel 62 is a converging outlet reservoir channel that converges toward the outlet reservoir 26, as illustrated in FIG. 3. The converging outlet reservoir channel can converge substantially continuously toward the outlet reservoir 26, or the converging outlet reservoir channel can include step changes that overall result in the converging toward the inlet reservoir 26. Although the inlet reservoir channel 60 and the outlet reservoir channel 62 are illustrated as being similar to each other in FIG. 3, it is to be understood that the configuration of the inlet reservoir channel 60 and the outlet reservoir channel 62 can be different from each other, within a same cell culture layer 22.

Either one of channels of the plurality of inlet reservoir channels 54, the plurality of outlet reservoir channels 58, the inlet reservoir channel 60 and the outlet reservoir channel 62 can include at least one turbulence reducing feature. A turbulence reducing feature can include a step change as described above in reference to the converging of the channel 28 toward the inlet reservoir 24 or the outlet reservoir 26. For instance, FIG. 3 illustrates an inlet reservoir channel 60 that includes a series of step changes, on the left-hand side of the cell culture layer 22.

### Fluid Medium Distribution System

In some implementations, an inlet fluid medium feeding system can be provided to supply an inlet fluid to the inlet well 48, and/or an outlet fluid medium feeding system can be provided to supply an outlet fluid to the outlet well 50. In some implementations, the inlet fluid medium feeding system and the outlet fluid medium feeding system can be combined and provided as a fluid medium feeding system, supplying both an inlet fluid to the inlet well 48, and an outlet fluid to the outlet well 50. The inlet fluid medium feeding system, outlet fluid medium feeding system or fluid medium feeding system can be any suitable structure configured to direct the introduction of a fluid medium into a corresponding well of the cell culture layer 22, and optionally contain a certain volume of fluid medium therein. In some implementations, the inlet fluid medium feeding system, outlet fluid medium feeding system or fluid medium feeding system can include a tube in fluid communication with a corresponding one of the inlet well 48 or the outlet well 50. In some implementations, the inlet fluid medium feeding system, outlet fluid medium feeding system or fluid medium feeding system can include an automated distribution system configured to provide a fluid medium to a corresponding one of the inlet well 48 or the outlet well 50 at given timepoints.

### Cover

In some implementations and as mentioned above, a cover 66 can be deposited on a top surface 27 of the cell culture layer 22, such as shown in FIGS. 4 and 5 for example. The cover 66 can be a biological model, i.e., a biologically active material, or a biologically inert material, depending on the in vitro model that is desired to achieve. When the cover 66 includes a biologically active material, the placement of the cover 66 on the top surface 27 of the cell culture layer 22 can enable interaction of the biological model with the biological material that is within the inlet reservoir 24, the outlet reservoir 26, and/or the channels 28. For a biological interaction between the inlet reservoir 24 and the biological model to occur, the inlet reservoir 24 can include at least some openings on a top surface thereof, or the inlet reservoir 24 can be an open-top inlet reservoir. Similarly, for a biological interaction between the outlet reservoir 26 and the biological model to occur, the outlet reservoir 26 can include at least some openings on a top surface thereof, or the outlet reservoir 26 can be an open-top outlet reservoir. For a biological interaction between the channels 28 and the biological model to occur, the channels 28 can include at least some openings on a top surface thereof, or the channels 28 can be open-top channels.

In the implementation illustrated in FIGS. 22-26, when the inlet reservoir 24 and the outlet reservoir 26 are placed in a superposed relationship with one or more channels extending therebetween, the cover 66 can be placed on the top surface 27 of the cell culture layer 22, and a biological interaction can occur between a biological material received in the inlet reservoir 24 and the biological model of the cover.

In some implementations, the biological model can be a three-dimensional skin model. A three-dimensional skin model can enable interaction with the elongated component of the biological material that is received in the channels 28, such as axons or hair, for instance. In some implementations, for instance when the biological model includes skin cells, the cover 66 can also include a collagen membrane. The cover 66 can also be made of any suitable biological tissue such as the intestines, muscles, the cornea, tumors, the heart, etc. As noted above, the biological model can also be cultured cells of such biological tissues. It is noted that the biological model can also be referred to as a three-dimensional biological tissue model.

In some implementations, when the biological tissue is a three-dimensional skin model, the three-dimensional skin model can include various types of cells, and generally include keratinocytes, Merkel cells, Langerhans cells, and melanocytes. The three-dimensional skin model can be a scaffold-based 3D model, which can reproduce the mechanical structure and the functionally of primary biological tissue. In scaffold-based 3D models, cells are grown on a support scaffold. The support scaffold can be made of natural polymers, such as collagen, fibronectin, elastin, fibrin, silk, alginate, chitosan, fibrin, or GAGs. The support scaffold can also be made of synthetic polymers, such as poly(ε-caprolactone) (PCL), polylactic acid, polyglycolic acid, polylactic-co-glycolic acid (PLGA), polyhydroxybutyrate, and polyethers such as polyethylene glycol (PEG) or PEG co-polymers.

In some implementations, when the cover 66 is made of a biologically inert material, the cover 66 can be made of various materials such as glass or plastics. Examples of plastics include PS, PP, COP, COC, and PC, to name a few. The cover 66 can have for instance a thickness ranging from 0.04 mm to 5 mm, although other thicknesses are also possible depending on the intended use. The thickness of the cover 66 can be determined so as to facilitate gas exchange with cell culture incubator environments. The cover 66 can also be made of a porous material like a sieve, a microporous membrane, or a porous microfiber, which can also contribute to facilitate gas exchange with cell culture incubator environments.

The cover 66 can be configured to fully cover the openings on the cell culture layer 22 such as the inlet reservoir 24, the outlet reservoir 26, and the inlet well 48 and the outlet well 50 if present. Alternatively, the cover 66 can partially cover these openings. For example, the cover 66 can be configured to cover the inlet reservoir 24 and the outlet reservoir 26, while the inlet well 48 and/or the outlet well 50 can remain open to atmosphere. The above-mentioned scenarios are examples only and as such, the cover 66 can have any configuration that enables desired receptacles of the cell culture layer 22 to be covered or to remain open to atmosphere.

In the implementation shown in FIGS. 3 and 4, a cover 66 is placed onto the upper surface, or top surface 27, of the cell culture layer 22. The cover 66 is removable from the top surface 27 of the cell culture layer 22. In the implementation shown, the cover 66 is illustrated as being transparent, which can facilitate visualization of the elongated component of the biological material receiving within the channels 28 of the cell culture layer 22. It is to be understood, however, that in other implementations, the cover 66 can range from opaque to transparent, for instance depending on the material from which it is made.

### Combination of Cell Culture Layer and Electrode Layer

In some implementations, the cell culture device 20 can be configured to be used in combination with, or can include, an electrode or a group of electrodes such that the electrode or group of electrodes can be in contact, either direct or indirect, i.e., in electrical communication, with the biological material present in at least one of the inlet reservoir 24, the channels 28 and the outlet reservoir 26. The electrode or group of electrodes can take the form of an electrode layer that can be placed underneath the cell culture layer 22, or on the top surface 27 of the cell culture layer 22. The electrode layer can be in direct contact with the cell culture layer 22, or can be in indirect contact with the cell culture layer 22, for instance if a membrane is placed underneath the cell culture layer 22 and the electrode layer is placed between the membrane and the bottom wall 34 of the cell culture plate 20, or if a cover 66 is placed on the top surface 27 of the cell culture layer 22 and the electrode layer is received on top of the cover 66. In yet other implementations, the electrode layer can form part of the biological model such that the biological model grows around it. It is to be understood that any suitable sequence of the cell culture layer 22, membrane, cover and electrode layer can be implemented depending on the in vitro compartmentalized model that is desired to be obtained.

In some implementations, multiple electrode layers can be provided according to a combination of any of the locations described above, e.g., under the cell culture layer, under the biological model, within the biological model, or above the biological model.

It is to be understood that the electrode layer can be provided in proximity of the channels 28 of the cell culture layer 22 and/or of the biological model, either directly or indirectly in contact therewith. The proximity of the electrode layer with the biological material being cultured in the cell culture layer 22 or the cells of the biological model can contribute to improving the detection and stimulation of the cells. When the electrode layer is provided in proximity of the cells, the distance between the electrode(s) of the electrode layer and the cells can be in the range of micrometers or millimeters, for instance.

In some implementations, the electrode can be configured to provide an electrical signal to stimulate cells growing in the inlet reservoir 24, in the channels 28 and/or in the outlet reservoir 26, or the cells of the biological model. The electrode can also be configured to collect, and/or record, and/or measure, and/or detect the response of cells to stimulation. In some implementations, the same electrode can be configurable to sequentially perform different actions. For instance, the electrode can be configured to collect a signal at a given timepoint, and at a subsequent timepoint, the electrode can be configured to provide an electrical signal. In some implementations, the electrode can be configured to detect an optical signal or an electrical signal.

In some implementations, the distribution of the electrodes over the surface area of the electrode layer can be such that it corresponds at least partially to the spatial distribution of the channels 28 of the cell culture layer 22, or vice versa, instead of the electrodes being provided randomly relative to the spatial distribution of the channels 28 of the cell culture layer 22. Providing the electrodes in such a configuration can enable obtaining electrodes in an organized fashion which in turn, can enable to better target the function of the electrodes over a controlled architecture and/or number of cells that are growing in the inlet reservoir 24, the channels 28, and/or the outlet reservoir 26. For instance, when neurons are cultured using the cell culture device as described herein, with cell bodies being received in the inlet reservoir or in the channel and axons extending within the channels, electrodes that are provided according to a controlled architecture determined at least in part according to the configuration of the channels can contribute to improve the stimulation of the neuronal cells or the detection of a signal from the neuronal cells.

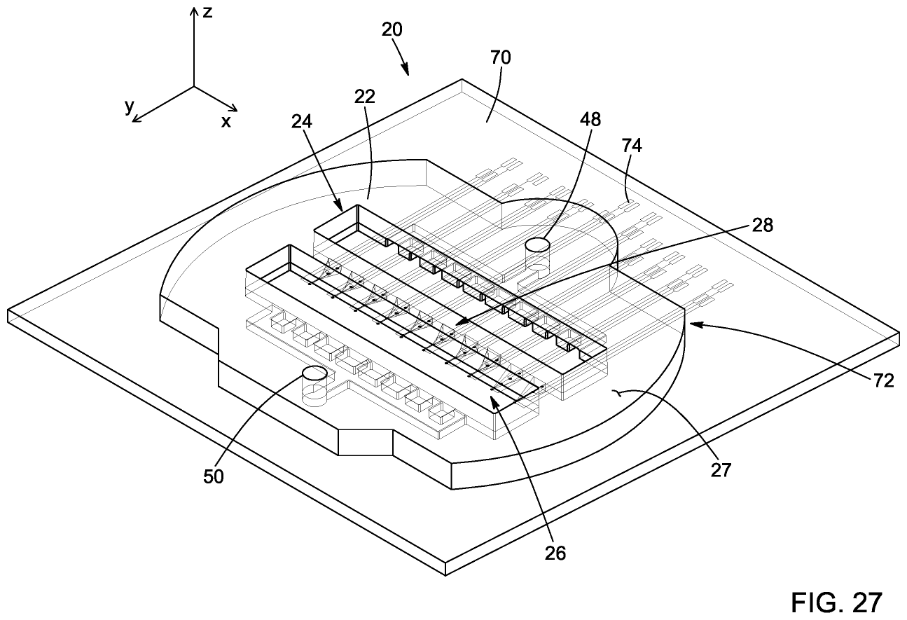

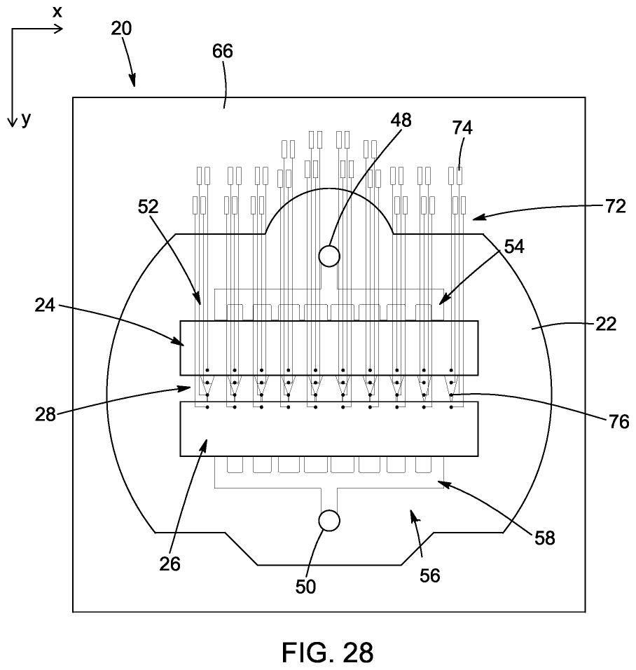

FIGS. 27 and 28 illustrate an example of a cell culture layer 22 that is used in combination with an electrode layer 70. In the implementation shown in FIGS. 27 and 28, the electrode layer 70 is provided underneath the cell culture layer 22. The electrode layer 70 includes electrodes 72 electrically connected by wires to a series of terminals 74. The terminals 74 are provided in proximity of the outer periphery of the cell culture layer 22, which can facilitate the interfacing of the terminals 74 with external equipment configured for reading signals from the electrodes 72. In the implementation shown in FIGS. 27 and 28, the tips 76 of each electrode 72 are aligned with a corresponding channel 28 extending between the inlet reservoir 24 and the outlet reservoir 26. In this example implementation, four electrodes are shown as being aligned with the corresponding channel 28, with a first one of the four electrodes being located in the inlet reservoir 24, at the entry of the corresponding channel 28, and a fourth one of the electrodes being located in the outlet reservoir 26, at the exit of the corresponding channel 28. It is to be understood that less than four electrodes or more than four electrodes can also be provided in the electrode layer as being associated with a corresponding channel. In addition, the number of electrodes associated with a given portion of the corresponding channel can vary with respect to another given portion of the corresponding channel, such that the electrodes are provided according to a given sequence in terms of number and distance from each other along the length of the corresponding channel to achieve a given pattern. Furthermore, the spatial distribution of the electrodes can vary from the illustrated implementation. For instance, multiple electrodes 72 and associated tips 76 can be provided in the inlet reservoir 24 and/or in the outlet reservoir 26, at variable distances from the channels 28. These multiple electrodes 72 can be provided according to an organized spatial distribution in either one of the inlet reservoir 24 or the outlet reservoir 26, or both. Although the electrode layer 70 illustrated in FIGS. 27 and 28 is shown in association with the cell culture layer 22 of FIG. 1, it is to be understood that the configuration of the electrode layer 70 can be adapted so that the electrode layer 70 can be used in combination with any other implementation of the cell culture layer 22 described herein.

In some implementations, the electrode can comprise at least one metallic electrode, at least one metal oxide electrode, at least one carbon electrode, a multi-electrode array, and/or at least one field effect transistor detectors.

In some implementations, the cell culture device can include any other types of sensors that can stimulate cells or measure responses of cells to stimulation. Examples of sensors can include optical sensors, chemical sensors, and electrical sensors, for instance.

In some implementations, the cell culture device can include an electrode set provided proximate to a biological material, which can be for instance the biological material cultured in the inlet reservoir, the channels and/or the outlet reservoir, and/or the biological model used in combination with the cell culture layer. The electrode set can include at least one electrode configured to collect an electric signal associated with at least a portion of the biological material. The electrode set can take the form of an electrode layer as described above, or can take a different form. The electrode set can include more or more electrodes. The electrodes can enable providing electrical read-outs comprising one or more of potential recordings, impedance spectroscopy, voltammetry and amperometry.

In some implementations, the cell culture device can include an electronic device in ohmic connection with the electrode described above. The electronic device can include for instance a sensing device or a stimulating device, and can be configured for providing electrical read-outs comprising one or more of potential recordings, impedance spectroscopy, voltammetry and amperometry. The electronic device can be located within a cell culture plate into which is received the cell culture layer, or be provided in proximity thereof.

In some implementations, when the biological material includes neuronal cells, the cell culture device can include a sensor configured for stimulating the neuronal cells, measuring a response from the neuronal cells to stimulation, providing an outlet and/or receiving an inlet. The sensor can include for instance an optical or an electrical transducer.

### Method for Preparing a Compartmentalized In Vitro Model

The general steps of a method for preparing a compartmentalized in vitro model using a cell culture layer received in a cell culture plate will now be described in further detail.

The method detailed in the following paragraphs can be implemented using a cell culture device 20 as described herein, of which examples are shown in FIGS. 1-26.

In some implementations, the method for preparing a compartmentalized in vitro model can be used to model a biological material that includes a multicellular component that is a non-elongated component, and an elongated component. In the context of the present description, the use of the expression “non-elongated” and of the term “elongated” can be interpreted as relative terms when such components are compared to one another. For instance, when the biological material is a neurosphere, the cluster of neuronal cell bodies can be considered as a non-elongated multicellular component of the biological material, and the axons would be considered as elongated components of the biological material. Thus, the multicellular component of the biological material can have an aspect ratio that is different than 1:1, but the multicellular component is generally less elongated than the elongated component and therefore is considered as a non-elongated component. The differentiation between the non-elongated component and the elongated component of the biological material can also be made in relation to the size of the channel into which the elongated component of the biological material is intended to be inserted. For instance, in the case of a neurosphere as a biological material, the channels can be configured so as to prevent the cluster of neuronal cell bodies to enter the channels or the elongated component receiving portion of the channel, and can rather be sized to selectively enable the axons to enter the channel or the elongated component receiving portion of the channel, resulting in the axons being considered as the elongated component and the cluster of neuronal cell bodies as the non-elongated component.

In some implementations, the method for preparing a compartmentalized in vitro model can be used to model a first biological material having a multicellular component, and a second biological material, the second biological material comprising an elongated component biologically interacting with the first biological material. In this scenario, the first biological material can be for instance follicles, and the second biological material can comprise hair. As mentioned above, the reference to an “elongated component” can be a relative term to compare the configuration of the elongated component versus the configuration of the first biological material, that is less elongated than the elongated component of the second biological material. The differentiation of the first biological material and the elongated component of the second biological material can also be made in relation to the size of the channel into which the elongated component of the second biological material is intended to be inserted. In other words, the channel or the elongated component receiving portion of the channel can be generally configured to limit the entry of the first biological material therein, but can be sized to receive the elongated component of the second biological material therein.

The method can include supplying a fluid culture medium to an inlet reservoir of the cell culture layer. When the cell culture device includes an inlet well and a manifold, or an inlet well and an inlet reservoir channel, the fluid culture medium can be supplied to the inlet well to successively travel from the inlet well to the inlet reservoir.

The multicellular component of the biological material can then be positioned within the inlet reservoir or within the multicellular component receiving portion of the channel of the cell culture layer.

When the multicellular component of the biological material is positioned within the inlet reservoir, at least a portion of the elongated component of the biological material can be inserted, or can grow, into a channel of the cell culture layer that is in fluid communication with the inlet reservoir. For instance, when the biological material includes a neurosphere, the cluster of neuronal cell bodies can be received in the inlet reservoir and/or in a portion of the channel that corresponds to a multicellular component receiving portion, and the axons can then grow into the channel (if the cluster of neuronal cell bodies is received in the inlet reservoir) or the portion of the channel that corresponds to an elongated component receiving portion (if the cluster of neuronal cell bodies is received in the portion of the channel that corresponds to a multicellular component receiving portion).

When at least a portion of the multicellular component of the biological material is positioned within the multicellular component receiving portion of the channel, at least a portion of the elongated component of the biological material can be inserted, or can grow, into the elongated component receiving portion of the channel provided downstream of the multicellular component receiving portion. For instance, when the biological material includes a neurosphere, the cluster of neuronal cell bodies can be received in the multicellular component receiving portion of the channel, and the axons can then grow into the elongated component receiving portion of the channels of the cell culture layer.

It is to be understood that more than one axon can grow into a single elongated component receiving portion, or a single axon can grow into a corresponding elongated component receiving portion.

Alternatively, a first biological material that includes a multicellular component can be positioned in the inlet reservoir of the cell culture layer, and a second biological material can be inserted or can grow into a channel of the cell culture layer that is in fluid communication with the inlet reservoir, the second biological material comprising an elongated component biologically interacting with the first biological material. For example, the first biological material can be follicles as a multicellular component, the follicles being positioned in the inlet reservoir or being received within a multicellular component receiving portion of a channel, and the hairs can be the elongated component of the second biological material that can be inserted or grown into the elongated component receiving portion of the channels of the cell culture layer.

It is to be understood that more than one hair can be inserted into a single elongated component receiving portion, or a single hair can be inserted into a corresponding elongated component receiving portion.

In both scenarios, the channels are configured to maintain the elongated component in a substantially elongated configuration. As described above, the channel can have various features to enable the maintaining of the elongated component in the substantially elongated configuration. For instance, the channel can include a converging portion that converges away from the inlet reservoir, i.e., that converges toward the outlet reservoir when the outlet reservoir is present. The channel can also include a series of inwardly protruding members that can contribute to maintain the elongated member in an elongated configuration within the channel. In some implementations, the series of inwardly protruding members can define arrowhead features that converge toward the outlet reservoir. The channel can include a neck portion. In some implementations, the diameter or width of the channel can be sufficient on its own to maintain the elongated component in the substantially elongated configuration.

The elongated component of the biological material or of the second biological material can extend within the outlet reservoir, when present. The presence of the elongated component in the outlet reservoir can enable contacting the elongated component with a test substance, as will be described in more detail below. In other implementations, the outlet reservoir can be omitted, and the elongated component can terminate within the channel.

When the outlet reservoir is present, the method can include supplying a fluid culture medium to the outlet reservoir of the cell culture layer. When the cell culture device includes an outlet well and a manifold, or an outlet well and an outlet reservoir channel, the fluid culture medium can be supplied to the outlet well to successively travel from the outlet well to the outlet reservoir.

The biological material can be cultured during a certain period of time in the cell culture layer, and subsequently be subjected to testing, or alternatively, the biological material can be subjected to testing without a given period of time dedicated to culturing the biological material. In some implementations, an inlet test substance can be added to the inlet reservoir to perform testing on the non-elongated component of the biological material, or on the first biological material, whichever is present in the inlet reservoir or in the multicellular component receiving portion. In some implementations, an outlet test substance can be added to the outlet reservoir to perform testing on the portion of the elongated component that extends within the outlet reservoir or within the elongated component receiving portion, or on the additional biological material that is present in the outlet reservoir or within the elongated component receiving portion and that is biologically interacting with the elongated component present in the channel.

The inlet test substance and the outlet test substance can be the same or different. The configuration of the cell culture layer as described herein can advantageously enable to produce compartmentalized in vitro models and to test distinct substances on given portions of a biological material or on given biological materials if more than one is present.

For instance, when the first biological material includes follicles and the elongated component of the second biological material includes hairs, the presence of the inlet reservoir can enable adding an inlet test substance to the inlet reservoir, contacting the follicles with the inlet test substance, and subsequently analyzing the response of the follicles to that inlet test substance. As at least a portion of the hairs are extending within the outlet reservoir, an outlet test substance can be added to the outlet reservoir to contact the at least a portion of the hairs, and the response of the hairs to that outlet test substance can subsequently be analyzed. When the follicles and the hairs are each contacted with their respective test substance, the interaction of the follicles and the hairs when contacted with their respective test substance can also be analyzed.

In accordance with another example, a multicellular component of a biological material such as a neurosphere can be placed in an inlet reservoir, an elongated component of the biological material, such as axons of neurons, can grow into the channel, and an additional biological material can be received within an outlet reservoir, and the presence of the inlet reservoir can enable adding an inlet test substance to the inlet reservoir, contacting the multicellular component with the inlet test substance, and subsequently analyzing the response of the multicellular component to that inlet test substance. An outlet test substance can be added to the outlet reservoir to contact the additional biological material, and the response of the additional biological material to that outlet test substance can subsequently be analyzed. When the multicellular component of the biological material and the additional biological material are each contacted with their respective test substance, the interaction of the multicellular component of the biological material and the additional biological material when contacted with their respective test substance can also be analyzed.

### Additional Examples of Applications for a Compartmentalized In Vitro Model

The compartmentalized in vitro models that can be obtained according to the techniques described herein can result in in vitro models having a tridimensional structural organization that more closely resemble the ones observed physiologically, which in turn can contribute to improving disease modelling, efficacy and toxicology testing, providing more accurate and reproducible physiologic responses to the substances being tested, and facilitating producing compartmentalized in vitro models that are more easily reproducible from batch-to-batch preparations.

The cell culture device as described herein can enable testing of substances with different viscosity such as oils, creams and cell medium, different solubility, such as oil and water-based compounds in one or both compartments.

Several alternative implementations and examples have been described and illustrated herein. The implementations of the technology described above are intended to be exemplary only. A person of ordinary skill in the art would appreciate the features of the individual implementations, and the possible combinations and variations of the components. A person of ordinary skill in the art would further appreciate that any of the implementations could be provided in any combination with the other implementations disclosed herein. It is understood that the technology may be embodied in other specific forms without departing from the central characteristics thereof. The present implementations and examples, therefore, are to be considered in all respects as illustrative and not restrictive, and the technology is not to be limited to the details given herein. Accordingly, while the specific implementations have been illustrated and described, numerous modifications come to mind.

1. A cell culture device for preparing a compartmentalized in vitro model using a biological material having a multicellular component and an elongated component, the cell culture device comprising:
a cell culture layer couplable to a reservoir of a cell culture plate, the cell culture layer comprising:
an inlet reservoir configured for receiving an inlet fluid medium therein;
an outlet reservoir configured for receiving an outlet fluid medium therein; and
a channel extending between the inlet reservoir and the outlet reservoir to establish fluid communication therebetween, the channel comprising:
a multicellular component receiving portion sized and configured for receiving at least a portion of the multicellular component of the biological material; and
an elongated component receiving portion provided downstream of the multicellular component receiving portion, the elongated component receiving portion being sized and configured for orienting the elongated component of the biological material away from the inlet reservoir and for preventing the multicellular component from travelling past the multicellular component receiving portion.

2. The cell culture device of claim 1, wherein the channel comprises a converging portion that converges inwardly toward the outlet reservoir.

3. The cell culture device of claim 2, wherein the converging portion defines at least a portion of the multicellular component receiving portion of the channel.

4. The cell culture device of claim 2 or 3, wherein the converging portion defines at least a portion of the elongated component receiving portion of the channel.

5. The cell culture device of any one of claims 2 to 4, wherein the converging portion includes an inwardly converging top wall that converges inwardly toward a center of the channel.

6. The cell culture device of claim 5, wherein the inwardly converging top wall defines a step change.

7. The cell culture device of claim 5, wherein the inwardly converging top wall includes a curvature.

8. The cell culture device of any one of claims 5 to 7, wherein the channel has a width that is substantially constant.

9. The cell culture device of any one of claims 2 to 4, wherein the converging portion includes an inwardly converging sidewall that converges inwardly toward a center of the channel.

10. The cell culture device of claim 9, wherein the inwardly converging sidewall defines a step change.

11. The cell culture device of claim 9, wherein the inwardly converging sidewall includes a curvature.

12. The cell culture device of any one of claims 2 to 4, wherein the converging portion comprises a frustoconical converging portion.

13. The cell culture device of any one of claims 2 to 4, wherein the converging portion comprises a frustopyramidal converging portion.

14. The cell culture device of any one of claims 2 to 13, wherein the converging portion comprises a neck portion to contribute to stabilizing the elongated component of the biological material.

15. The cell culture device of any one of claims 2 to 14, wherein the converging portion comprises an inwardly protruding member to contribute to stabilizing the elongated component of the biological material.

16. The cell culture device of any one of claims 2 to 15, wherein the channel further comprises a tubular portion provided downstream of the converging portion, the tubular portion having a substantially constant diameter throughout its length.

17. The cell culture device of any one of claims 2 to 16, wherein the converging portion of the channel comprises converging sections each converging at a corresponding angle toward the outlet reservoir.

18. The cell culture device of claim 17, wherein the converging sections successively comprises a first converging section and a second converging section, the corresponding angle of the first converging section being larger than the corresponding angle of the second converging section.

19. The cell culture device of claim 17 or 18, wherein a transition between successive ones of the converging sections comprises a curved transition.

20. The cell culture device of claim 17 or 18, wherein a transition between successive ones of the converging sections comprises a sharp transition.

21. The cell culture device of any one of claims 1 to 20, wherein the channel is sized and configured to maintain the elongated component of the biological material in a substantially elongated configuration.

22. The cell culture device of any one of claims 1 to 21, wherein the channel comprises a plurality of channels.

23. The cell culture device of claim 22, wherein adjacent channels of the plurality of channels are similar to each other.

24. The cell culture device of claim 22, wherein at least one channel of the plurality of channels is configured differently than at least one other channel of the plurality of channels.

25. The cell culture device of any one of claims 22 to 24, wherein the inlet reservoir comprises a plurality of inlet reservoirs.

26. The cell culture device of claim 25, wherein at least one channel of the plurality of channels is in fluid communication with a corresponding inlet reservoir of the plurality of inlet reservoirs.

27. The cell culture device of any one of claims 22 to 26, wherein the outlet reservoir comprises a plurality of outlet reservoirs.

28. The cell culture device of claim 27, wherein at least one channel of the plurality of channels is in fluid communication with a corresponding outlet reservoir of the plurality of outlet reservoirs.

29. The cell culture device of any one of claims 1 to 28, wherein the cell culture layer is configured to extend substantially horizontally, the inlet reservoir and the outlet reservoir being provided in a longitudinally spaced-apart relationship.

30. The cell culture device of claim 29, wherein the cell culture layer further comprises an inlet well in fluid communication with the inlet reservoir, the inlet well being configured to receive the inlet fluid medium therein.

31. The cell culture device of claim 30, wherein the cell culture layer comprises an inlet manifold extending between the inlet well and the inlet reservoir, the inlet manifold comprising a plurality of inlet reservoir channels for directing flow of the inlet fluid medium from the inlet well to the inlet reservoir with reduced turbulence.

32. The cell culture device of claim 31, wherein the cell culture layer comprises an inlet reservoir channel extending between the inlet well and the inlet reservoir for directing flow of the inlet fluid medium into the inlet reservoir with reduced turbulence.

33. The cell culture device of claim 32, wherein the inlet reservoir channel is a converging inlet reservoir channel converging toward the inlet reservoir.

34. The cell culture device of claim 32 or 33, wherein the inlet reservoir channel comprises at least one turbulence reducing feature.

35. The cell culture device of any one of claims 29 to 34, wherein the cell culture layer further comprises an outlet well in fluid communication with the outlet reservoir, the outlet well being configured to receive the outlet fluid medium therein.

36. The cell culture device of claim 35, wherein the cell culture layer comprises an outlet manifold extending between the outlet reservoir and the outlet well, the outlet manifold comprising a plurality of outlet reservoir channels for directing flow of the outlet fluid medium from the outlet reservoir to the outlet well with reduced turbulence.

37. The cell culture device of claim 35, wherein the cell culture layer comprises an outlet reservoir channel extending between the outlet reservoir and the outlet well for directing flow of the outlet fluid medium from the outlet reservoir to the outlet well with reduced turbulence.

38. The cell culture device of claim 37, wherein the outlet reservoir channel is a converging outlet reservoir channel converging toward the outlet well.

39. The cell culture device of claim 37 or 38, wherein the outlet reservoir channel comprises at least one turbulence reducing feature.

40. The cell culture device of any one of claims 9 to 20, wherein the cell culture layer is configured to extend substantially horizontally, the inlet reservoir and the outlet reservoir being provided in a superposed relationship, and the converging portion being a downwardly converging portion.

41. The cell culture device of claim 40, wherein the outlet reservoir is U-shaped.

42. The cell culture device of claim 40 or 41, wherein the inlet reservoir comprises a plurality of inlet reservoirs, and adjacent ones of the plurality of inlet reservoirs are in fluid communication via an inlet reservoir bridge extending therebetween.

43. The cell culture device of any one of claims 1 to 39, further comprising a cover configured to be positionable on an upper surface of the cell culture layer.

44. The cell culture device of claim 43, wherein the cover is configured to provide a fluid tight closure for the inlet reservoir once positioned on the upper surface of the cell culture layer.

45. The cell culture device of claim 43 or 44, wherein the cover comprises a microporous membrane.

46. The cell culture device of claim 43 or 44, wherein the cover comprises a collagen membrane.

47. The cell culture device of any one of claims 1 to 39, further comprising a biological model positionable on an upper surface of the cell culture layer.

48. The cell culture device of claim 47, wherein the biological model is positionable on the cell culture layer to enable interaction with the biological material received in the channel.

49. The cell culture device of claim 47 or 48, wherein the biological model comprises cultured cells.

50. The cell culture device of claim 47 or 48, wherein the biological model comprises a biological tissue.

51. The cell culture device of claim 47 or 48, wherein the biological model comprises a biological tissue model.

52. The cell culture device of claim 51, wherein the biological tissue model comprises a three-dimensional skin model.

53. The cell culture device of any one of claims 1 to 52, further comprising an inlet well feeding system in fluid communication with the inlet well to supply additional fluid culture medium to the inlet well.

54. The cell culture device of any one of claims 1 to 53, wherein the cell culture plate is a petri dish.

55. The cell culture device of any one of claims 1 to 54, wherein the cell culture device complies with American National Standards Institute of the Society for Laboratory Automation and Screening (ANSI/SLAS) microplate standards.

56. The cell culture device of any one of claims 1 to 55, wherein the multicellular component of the biological material comprises a cluster of neuronal cell bodies and the elongated component comprising axons.

57. The cell culture device of any one of claims 1 to 55, wherein the multicellular component of the biological material comprises a follicle and the elongated component comprising a hair.

58. A cell culture device for preparing a compartmentalized in vitro model using a biological material having a multicellular component and an elongated component, the cell culture device comprising:
a cell culture layer insertable in a reservoir of a cell culture plate, the cell culture layer comprising:
an inlet reservoir configured for receiving an inlet fluid medium therein;
an outlet reservoir configured for receiving an outlet fluid medium therein; and
a channel extending between the inlet reservoir and the outlet reservoir to establish fluid communication therebetween, the channel comprising:
a multicellular component receiving portion sized and configured for receiving at least a portion of the multicellular component of the biological material; and
an elongated component receiving portion provided downstream of the multicellular component receiving portion, the elongated component receiving portion being sized and configured for orienting the elongated component of the biological material away from the inlet reservoir and for preventing the multicellular component from travelling past the multicellular component receiving portion; and
an electrode provided in proximity of the cell culture layer.

59. The cell culture device of claim 58, wherein the electrode forms part of an electrode layer.

60. The cell culture device of claim 58, wherein the electrode layer is positionable underneath the cell culture layer.

61. The cell culture device of claim 58, wherein the electrode layer is receivable onto an upper surface of the cell culture layer.

62. The cell culture device of claim 59, further comprising a biological model receivable on an upper surface of the cell culture layer.

63. The cell culture device of claim 62, wherein the electrode layer is provided onto the biological model.

64. The cell culture device of claim 62, wherein the electrode layer is provided as part of the biological model.

65. The cell culture device of any one of claims 58 to 64, wherein the electrode comprises a plurality of electrodes.

66. The cell culture device of claim 65, wherein the channel comprises a plurality of channels.

67. The cell culture device of claim 65, wherein the plurality of electrodes are distributed over the electrode layer in accordance with a configuration of the plurality of channels.

68. The cell culture device of claim 58, wherein the electrode is located in an adjacent reservoir.

69. The cell culture device of any one of claims 58 to 68, wherein the electrode comprises at least one of a metallic electrode, a metal oxide electrode, a carbon electrode, a multi-electrode array, and a field effect transistor detector.

70. The cell culture device of any one of claims 58 to 69, wherein the electrode is configured for stimulating the biological material.

71. The cell culture device of any one of claims 58 to 70, wherein the electrode is configured for at least one of collecting, recording, measuring, or detecting a response of the biological material to stimulation.

72. The cell culture device of any one of claims 58 to 71, further comprising an electronic device in ohmic connection with the electrode.

73. The cell culture device of claim 72, wherein the electronic device comprises a sensing device.

74. The cell culture device of claim 72, wherein the electronic device comprises a stimulating device.

75. The cell culture device of any one of claims 72 to 74, wherein the electronic device is configured for providing an electrical read-out comprising at least one of a potential recording, an impedance spectroscopy recording, a voltammetry recording and an amperometry recording.

76. The cell culture device of any one of claims 72 to 75, further comprising a sensor configured for stimulating the biological material, measuring a response from the biological material to stimulation, providing an outlet or receiving an inlet.

77. The cell culture device of claim 76, wherein the sensor comprises an optical or an electrical transducer.

78. A method for preparing a compartmentalized in vitro model of a biological material that includes a multicellular component and an elongated component using a cell culture layer receivable in a cell culture plate, the method comprising:
supplying a fluid culture medium to an inlet reservoir of the cell culture layer;
positioning the multicellular component of the biological material within the inlet reservoir of the cell culture plate; and
inserting and/or growing at least a portion of the elongated component of the biological material into a channel of the cell culture layer that is in fluid communication with the inlet reservoir, the channel comprising a multicellular component receiving portion and an elongated component receiving portion provided downstream of the multicellular component receiving portion, the elongated component receiving portion being sized and configured for orienting the elongated component of the biological material away from the inlet reservoir and for preventing the multicellular component from travelling past the multicellular component receiving portion.

79. The method of claim 78, wherein the channel comprises a converging portion that converges inwardly.

80. The method of claim 78 or 79, wherein the cell culture layer further comprises an outlet reservoir in fluid communication with the channel, with the channel extending between the inlet reservoir and the outlet reservoir.

81. The method of claim 80, wherein at least a portion of the elongated component of the biological material extend within the outlet reservoir.

82. The method of claim 81, further comprising adding an outlet test substance to the outlet reservoir to perform testing on the at least a portion of the elongated component of the biological material.

83. The method of claim 80 or 81, further comprising positioning an additional biological material within the outlet reservoir.

84. The method of claim 83, further comprising adding an outlet test substance to the outlet reservoir to perform testing on additional biological material.

85. The method of any one of claims 78 to 84, further comprising adding an inlet test substance to the inlet reservoir to perform testing on the non-elongated component of the biological material.

86. The method of any one of claims 78 to 85, further comprising placing a cover on an upper surface of the cell culture layer.

87. The method of claim 86, wherein the cover comprises a microporous membrane.

88. The method of claim 86, wherein the cover comprises a collagen membrane.

89. The method of any one of claims 78 to 85, further comprising placing a biological model on an upper surface of the cell culture layer.

90. The method of claim 89, wherein the biological model is positionable on the cell culture layer to enable interaction with the biological material received in the cell culture layer.

91. The method of any one of claims 78 to 90, wherein the multicellular component of the biological material comprises follicles.

92. The method of claim 91, wherein the elongated component of the biological material comprises hair.

93. The method of any one of claims 78 to 90, wherein the multicellular component of the biological material comprises cell bodies of neurons.

94. The method of claim 93, wherein the biological material is provided as a neurosphere.

95. The method of claim 93 or 94, wherein the elongated component of the biological material comprises axons.

96. A method for preparing a compartmentalized in vitro model of a biological material using a cell culture layer receivable in a cell culture plate, the method comprising:
supplying a fluid culture medium to an inlet reservoir of the cell culture layer;
positioning a first biological material that includes a multicellular component within the inlet reservoir of the cell culture plate; and
inserting and/or growing a second biological material into a channel of the cell culture layer that is in fluid communication with the inlet reservoir, the second biological material comprising an elongated component biologically interacting with the first biological material, and the channel being configured to maintain the elongated component in a substantially elongated configuration.

97. The method of claim 96, wherein the channel comprises a converging portion that converges inwardly toward the outlet reservoir.

98. The method of claim 96 or 97, wherein the cell culture layer further comprises an outlet reservoir in fluid communication with the channel, with the channel extending between the inlet reservoir and the outlet reservoir.

99. The method of claim 98, wherein at least a portion of the second biological material extends within the outlet reservoir.

100. The method of claim 98 or 99, further comprising adding an outlet test substance to the outlet reservoir to perform testing on the second biological material.

101. The method of claim 98 or 99, further comprising positioning an additional biological material within the outlet reservoir.

102. The method of claim 101, further comprising adding an outlet test substance to the outlet reservoir to perform testing on the additional biological material.

103. The method of any one of claims 96 to 102, further comprising adding an inlet test substance to the inlet reservoir to perform testing on the first biological material.

104. The method of any one of claims 96 to 103, further comprising placing a cover on an upper surface of the cell culture layer.

105. The method of claim 104, wherein the cover comprises a microporous membrane.

106. The method of claim 104, wherein the cover comprises a collagen membrane.

107. The method of any one of claims 96 to 103, further comprising placing a biological model on an upper surface of the cell culture layer.

108. The method of claim 107, wherein the biological model is positionable on the cell culture layer to enable interaction with the biological material received in the cell culture layer.

109. The method of any one of claims 96 to 108, wherein the first biological material comprises neurons.

110. The method of claim 109, wherein the neurons are provided as neurospheres.

111. The method of claim 109 or 110, wherein the elongated component of the second biological material comprises axons.

112. The method of any one of claims 96 to 108, wherein the first biological material comprises follicles.

113. The method of claim 112, wherein the second biological material comprises hairs.

114. The method of claim 101 or 102, wherein the additional biological material comprises at least one of heart tissue, intestinal tissue, muscle tissue, and corneal tissue.

115. A cell culture device for preparing a compartmentalized in vitro model using a biological material having a multicellular component and an elongated component, the cell culture device comprising:
a cell culture layer couplable to a reservoir of a cell culture plate, the cell culture layer comprising:
an inlet reservoir configured for receiving an inlet fluid medium and the multicellular component of the biological material therein;
an outlet reservoir configured for receiving an outlet fluid medium therein; and
a channel extending between the inlet reservoir and the outlet reservoir to establish fluid communication therebetween, the channel being sized and configured for selectively retaining the multicellular component of the biological material upstream of the channel while the elongated component extends within the channel.

116. The cell culture device of claim 115, wherein the channel comprises a converging portion that converges inwardly toward the outlet reservoir.

117. The cell culture device of claim 116, wherein the converging portion includes an inwardly converging top wall that converges inwardly toward a center of the channel.

118. The cell culture device of claim 117, wherein the inwardly converging top wall defines a step change.

119. The cell culture device of claim 117, wherein the inwardly converging top wall includes a curvature.

120. The cell culture device of any one of claims 117 to 119, wherein the channel has a width that is substantially constant.

121. The cell culture device of claim 116, wherein the converging portion includes an inwardly converging sidewall that converges inwardly toward a center of the channel.

122. The cell culture device of claim 121, wherein the inwardly converging sidewall defines a step change.

123. The cell culture device of claim 121, wherein the inwardly converging sidewall includes a curvature.

124. The cell culture device of claim 116, wherein the converging portion comprises a frustoconical converging portion.

125. The cell culture device of claim 116, wherein the converging portion comprises a frustopyramidal converging portion.

126. The cell culture device of any one of claims 116 to 125, wherein the converging portion comprises a neck portion to contribute to stabilizing the elongated component of the biological material.

127. The cell culture device of any one of claims 116 to 126, wherein the converging portion comprises an inwardly protruding member to contribute to stabilizing the elongated component of the biological material.

128. The cell culture device of any one of claims 116 to 127, wherein the channel further comprises a tubular portion provided downstream of the converging portion, the tubular portion having a substantially constant diameter throughout its length.

129. The cell culture device of any one of claims 116 to 128, wherein the converging portion of the channel comprises converging sections each converging at a corresponding angle toward the outlet reservoir.

130. The cell culture device of claim 129, wherein the converging sections successively comprises a first converging section and a second converging section, the corresponding angle of the first converging section being larger than the corresponding angle of the second converging section.

131. The cell culture device of claim 129 or 130, wherein a transition between successive ones of the converging sections comprises a curved transition.

132. The cell culture device of claim 129 or 130, wherein a transition between successive ones of the converging sections comprises a sharp transition.

133. The cell culture device of any one of claims 115 to 132, wherein the channel is sized and configured to maintain the elongated component of the biological material in a substantially elongated configuration.

134. The cell culture device of any one of claims 115 to 133, wherein the channel comprises a plurality of channels.

135. The cell culture device of claim 134, wherein adjacent channels of the plurality of channels are similar to each other.

136. The cell culture device of claim 134, wherein at least one channel of the plurality of channels is configured differently than at least one other channel of the plurality of channels.

137. The cell culture device of any one of claims 134 to 136, wherein the inlet reservoir comprises a plurality of inlet reservoirs.

138. The cell culture device of claim 137, wherein at least one channel of the plurality of channels is in fluid communication with a corresponding inlet reservoir of the plurality of inlet reservoirs.

139. The cell culture device of any one of claims 134 to 138, wherein the outlet reservoir comprises a plurality of outlet reservoirs.

140. The cell culture device of claim 139, wherein at least one channel of the plurality of channels is in fluid communication with a corresponding outlet reservoir of the plurality of outlet reservoirs.

141. The cell culture device of any one of claims 115 to 140, wherein the cell culture layer is configured to extend substantially horizontally, the inlet reservoir and the outlet reservoir being provided in a longitudinally spaced-apart relationship.

142. The cell culture device of claim 141, wherein the cell culture layer further comprises an inlet well in fluid communication with the inlet reservoir, the inlet well being configured to receive the inlet fluid medium therein.

143. The cell culture device of claim 142, wherein the cell culture layer comprises an inlet manifold extending between the inlet well and the inlet reservoir, the inlet manifold comprising a plurality of inlet reservoir channels for directing flow of the inlet fluid medium from the inlet well to the inlet reservoir with reduced turbulence.

144. The cell culture device of claim 143, wherein the cell culture layer comprises an inlet reservoir channel extending between the inlet well and the inlet reservoir for directing flow of the inlet fluid medium into the inlet reservoir with reduced turbulence.

145. The cell culture device of claim 144, wherein the inlet reservoir channel is a converging inlet reservoir channel converging toward the inlet reservoir.

146. The cell culture device of claim 144 or 145, wherein the inlet reservoir channel comprises at least one turbulence reducing feature.

147. The cell culture device of any one of claims 141 to 146, wherein the cell culture layer further comprises an outlet well in fluid communication with the outlet reservoir, the outlet well being configured to receive the outlet fluid medium therein.

148. The cell culture device of claim 147, wherein the cell culture layer comprises an outlet manifold extending between the outlet reservoir and the outlet well, the outlet manifold comprising a plurality of outlet reservoir channels for directing flow of the outlet fluid medium from the outlet reservoir to the outlet well with reduced turbulence.

149. The cell culture device of claim 147, wherein the cell culture layer comprises an outlet reservoir channel extending between the outlet reservoir and the outlet well for directing flow of the outlet fluid medium from the outlet reservoir to the outlet well with reduced turbulence.

150. The cell culture device of claim 149, wherein the outlet reservoir channel is a converging outlet reservoir channel converging toward the outlet well.

151. The cell culture device of claim 149 or 150, wherein the outlet reservoir channel comprises at least one turbulence reducing feature.

152. The cell culture device of any one of claims 121 to 132, wherein the cell culture layer is configured to extend substantially horizontally, the inlet reservoir and the outlet reservoir being provided in a superposed relationship, and the converging portion being a downwardly converging portion.

153. The cell culture device of claim 152, wherein the outlet reservoir is U-shaped.

154. The cell culture device of claim 152 or 153, wherein the inlet reservoir comprises a plurality of inlet reservoirs, and adjacent ones of the plurality of inlet reservoirs are in fluid communication via an inlet reservoir bridge extending therebetween.

155. The cell culture device of any one of claims 115 to 151, further comprising a cover configured to be positionable on an upper surface of the cell culture layer.

156. The cell culture device of claim 155, wherein the cover is configured to provide a fluid tight closure for the inlet reservoir once positioned on the upper surface of the cell culture layer.

157. The cell culture device of claim 155 or 156, wherein the cover comprises a microporous membrane.

158. The cell culture device of claim 155 or 156, wherein the cover comprises a collagen membrane.

159. The cell culture device of any one of claims 115 to 151, further comprising a biological model positionable on an upper surface of the cell culture layer.

160. The cell culture device of claim 159, wherein the biological model is positionable on the cell culture layer to enable interaction with the biological material received in the channel.

161. The cell culture device of claim 159 or 160, wherein the biological model comprises cultured cells.

162. The cell culture device of claim 159 or 160, wherein the biological model comprises a biological tissue.

163. The cell culture device of claim 159 or 160, wherein the biological model comprises a biological tissue model.

164. The cell culture device of claim 163, wherein the biological tissue model comprises a three-dimensional skin model.

165. The cell culture device of any one of claims 115 to 164, further comprising an inlet well feeding system in fluid communication with the inlet well to supply additional fluid culture medium to the inlet well.

166. The cell culture device of any one of claims 115 to 165, wherein the cell culture plate is a petri dish.

167. The cell culture device of any one of claims 115 to 166, wherein the cell culture device complies with American National Standards Institute of the Society for Laboratory Automation and Screening (ANSI/SLAS) microplate standards.

168. The cell culture device of any one of claims 115 to 167, wherein the multicellular component of the biological material comprises a cluster of neuronal cell bodies and the elongated component comprising axons.

169. The cell culture device of any one of claims 115 to 167, wherein the multicellular component of the biological material comprises a follicle and the elongated component comprising a hair.

170. A method for preparing a compartmentalized in vitro model of a biological material that includes a multicellular component and an elongated component using a cell culture layer receivable in a cell culture plate, the method comprising:
supplying a fluid culture medium to an inlet reservoir of the cell culture layer;
positioning the multicellular component of the biological material within a multicellular component receiving portion of a channel of the cell culture layer, the channel being in fluid communication with the inlet reservoir; and
inserting and/or growing the elongated component of the biological material into an elongated component receiving portion of the channel located downstream of the multicellular component receiving portion, the elongated component receiving portion of the channel being sized and configured for preventing the multicellular component from travelling past the multicellular component receiving portion.

171. The method of claim 170, wherein the channel comprises a converging portion that converges inwardly.

172. The method of claim 170 or 171, wherein the cell culture layer further comprises an outlet reservoir in fluid communication with the channel, with the channel extending between the inlet reservoir and the outlet reservoir.

173. The method of claim 172, wherein at least a portion of the elongated component of the biological material extend within the outlet reservoir.

174. The method of claim 173, further comprising adding an outlet test substance to the outlet reservoir to perform testing on the at least a portion of the elongated component of the biological material.

175. The method of claim 172 or 173, further comprising positioning an additional biological material within the outlet reservoir.

176. The method of claim 175, further comprising adding an outlet test substance to the outlet reservoir to perform testing on additional biological material.

177. The method of any one of claims 170 to 176, further comprising adding an inlet test substance to the inlet reservoir to perform testing on the non-elongated component of the biological material.

178. The method of any one of claims 170 to 177, further comprising placing a cover on an upper surface of the cell culture layer.

179. The method of claim 178, wherein the cover comprises a microporous membrane.

180. The method of claim 178, wherein the cover comprises a collagen membrane.

181. The method of any one of claims 170 to 177, further comprising placing a biological model on an upper surface of the cell culture layer.

182. The method of claim 181, wherein the biological model is positionable on the cell culture layer to enable interaction with the biological material received in the cell culture layer.

183. The method of any one of claims 170 to 182, wherein the multicellular component of the biological material comprises follicles.

184. The method of claim 183, wherein the elongated component of the biological material comprises hair.

185. The method of any one of claims 170 to 182, wherein the multicellular component of the biological material comprises cell bodies of neurons.

186. The method of claim 185, wherein the biological material is provided as a neurosphere.

187. The method of claim 185 or 186, wherein the elongated component of the biological material comprises axons.
